(19) United States
(12) Patent Application Publication
(10) Pub. No.: US 2026/0083705 A1
(43) Pub. Date: Mar. 26, 2026

(54) TRANSDERMAL DEXMEDETOMIDINE FOR TREATING AGITATION

(71) Applicant: Teikoku Pharma USA, Inc., San Jose, CA (US)

(72) Inventors: Wan-Ning Song, San Jose, CA (US); Gary G. Kay, St. Petersburg Beach, FL (US)

(21) Appl. No.: 19/338,730

(22) Filed: Sep. 24, 2025

Related U.S. Application Data
(60) Provisional application No. 63/698,171, filed on Sep. 24, 2024.
(60) Provisional application No. 63/797,544, filed on Apr. 30, 2025.

(51) Int. Cl. A61K 31/4174; A61K 9/70; A61P 25/28

(52) U.S. Cl. CPC A61K 31/4174; A61K 9/7038; A61P 25/28

(57) ABSTRACT

The present disclosure relates to methods of using transdermal delivery patches containing dexmedetomidine to treat agitation.

### 1. BACKGROUND

Acute agitation is a severe, disruptive, and distressing complication of many chronic mental disorders, including dementia. Current standard of care treatment is pharmacological tranquilization with antipsychotics and/or benzodiazepines.

Igalmi™, a sublingually or buccally administered film containing 120 mcg or 180 mg of dexmedetomidine, was recently approved by the U.S. Food and Drug Administration (FDA) for the acute treatment of agitation associated with schizophrenia or bipolar I or II disorder in adults. Lower dosages are recommended for individuals that are geriatric (i.e., >65 years old) (120 mcg) or that have hepatic impairment (60-120 mcg depending on the level of agitation severity). The prescribing information for Igalmi™ indicates that, if agitation persists after the initial dose, up to two additional doses may be administered at least two hours apart.

In clinical studies (NCT04268303 and NCT04276883), Igalmi™ demonstrated a statistically greater mean change from baseline, compared to placebo, in the Positive and Negative Syndrome Scale-Excited Component (PEC) total score at two hours after the first dose in patients treated with 180 mcg and 120 mcg. Decreases in agitation were observed 20-30 minutes following administration of Igalmi™. The safety and effectiveness of Igalmi™ was not established beyond 24 hours from the first dose.

There is a need for alternative methods of treating and preventing agitation, e.g., methods of prophylactically minimizing or eliminating the frequency and/or severity of agitation in patients that have a history of agitation, or conditions characterized by episodic agitation. For such patients, e.g., those with chronic conditions like Alzheimer's disease, treatment should be safe and have sustained efficacy in reducing the frequency and severity of agitation over an extended (e.g., multi-day) duration.

### 2. SUMMARY

The present disclosure relates to transdermal delivery patches that provide sustained release of dexmedetomidine for use in the treatment of agitation.

In one aspect, a method for treating agitation in a subject in need thereof is provided, comprising applying one or more transdermal delivery patches to the skin surface of the subject while not manifesting acute agitation at the time of patch application (e.g., a subject who is not agitated at the time of application),
wherein the transdermal delivery patch comprises a pharmaceutical composition comprising dexmedetomidine; and
wherein the one or more transdermal delivery patches deliver a therapeutically effective, non-sedating amount of dexmedetomidine to the subject.

In certain embodiments, the agitation is associated with major neurocognitive disorder (MND), e.g., dementia of the Alzheimer's type.

In certain embodiments, the one or more transdermal delivery patches are maintained on the skin surface of the subject for a duration of at least 72 hours (e.g., 96 hours).

In certain embodiments, the frequency or severity of agitation in the subject is reduced following application of the one or more transdermal delivery patches and maintenance on the skin surface for at least 72 hours (e.g., 96 hours). In certain embodiments, reduction in frequency or severity of agitation behaviors is determined by a change (i.e., improvement) in one or more of the following scales used to measure agitation:
Agitated Behavior Scale (ABS),
Cohen Mansfeld Agitation Inventory (CMAI),
Clinical Global Impression (Severity CGI-S); and Change (CGI-C),
Neuropsychiatric Inventory-Nursing Home Version (NPI-NH) and
the Episodic Agitation Scale-Nursing Home (EAS-NH).

In certain embodiments, the one or more transdermal delivery patches provide a non-sedating reduction in agitation in the subject.

### 3. BRIEF DESCRIPTION OF THE DRAWINGS

These and other features, aspects, and advantages of the present disclosure will become better understood with regard to the following description, and accompanying drawings, where:

FIGS. 1A-1C show views of exemplary transdermal delivery patches of the present disclosure. FIG. 1A shows a top view and side view of an exemplary transdermal delivery patch. FIG. 1B shows a top view and dimensions of an exemplary 6 cm² transdermal delivery patch (denoted dimensions are in inches). FIG. 1C shows a top view and dimensions of exemplary 9 cm² and 12 cm² transdermal delivery patches (denoted dimensions are in inches). For FIG. 1B and FIG. 1C, the image on the left shows the dimensions of the transdermal delivery patch. The interior square with rounded corners corresponds to the adhesive transdermal delivery patch. The larger, outer square corresponds to the release liner dimensions. The image on the right shows the dimensions of the pouch for the transdermal delivery patch.

FIG. 2 shows the schedule of events for the clinical trial of Example 2, where days are defined as 24-hour intervals relative to the time of DMTS/matching placebo system application.

FIG. 3 shows the schedule of agitation assessments for the clinical trial of Example 2.

### 4. DETAILED DESCRIPTION

### 4.1 Definitions

Unless defined otherwise, all technical and scientific terms used herein have the same meaning as commonly understood by one of ordinary skill in the art to which this disclosure applies.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, “dexmedetomidine” refers to the S-enantiomer of medetomidine:

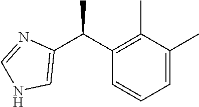

or a pharmaceutically acceptable salt, hydrate or complex of dexmedetomidine. Dexmedetomidine may be in the form of a pharmaceutically acceptable salt including, but not limited to, a mesylate, maleate, fumarate, tartrate, hydrochloride, hydrobromide, p-toluenesulfonate, benzoate, acetate, phosphate or sulfate salt. In certain embodiments, “dexmedetomidine” refers to the hydrochloride salt of dexmedetomidine, i.e., (+)-4-(S)-[1-(2,3-dimethylphenyl)ethyl]-1H-imidazole monohydrochloride.

Dexmedetomidine is a relatively selective alpha2-adrenergic agonist. Dexmedetomidine is the S-enantiomer of medetomidine. Precedex® (dexmedetomidine hydrochloride) is FDA approved for (1) sedation of initially intubated and mechanically ventilated patients during treatment in an intensive care setting and (2) sedation of non-intubated patients prior to and/or during surgical and other procedures. Precedex® is administered by continuous infusion.

As used herein, “dosage interval” refers to the period of time the dexmedetomidine transdermal delivery patches described herein are maintained in contact with the skin surface of the subject.

As used herein, “non-sedative” refers to an amount of dexmedetomidine that does not cause complete sedation of the subject. Suitable protocols for determining level of sedation may include, but are not limited to, the Ramsay Sedation Scale, the Vancouver Sedative Recovery Scale, the Glasgow Coma Scale modified by Cook and Palma, the Comfort Scale, the New Sheffield Sedation Scale, the Sedation-Agitation Scale, and the Motor Activity Assessment Scale, and the Wilson Sedation Score. In certain embodiments, the level of sedation is evaluated using the Wilson Sedation Scale, details of which are available at the website produced by placing “http://www.” prior to “sedationsolutions.co.uk/training-series6.php” and below:

The Wilson Sedation Score:
1. Fully awake and oriented
2. Drowsy
3. Eyes closed but rousable to command
4. Eyes closed but rousable to mild physical stimulation (earlobe tug)
5. Eyes closed but unrousable to mild physical stimulation

As used herein, “subject” refers to the person or organism to which the transdermal delivery patch is applied. As such, subjects of the invention may include but are not limited to mammals, e.g., humans and other primates, such as chimpanzees and other apes and monkey species; and the like, where in certain embodiments the subject are humans. The term subject is also meant to include a person or organism of any age, weight, or other physical characteristic, where the subjects may be an adult, an adolescent, a child, an infant or a newborn.

As used herein, “transdermal” refers to the route of administration where an active agent (i.e., drug) is delivered across the skin (e.g., topical administration) or mucous membrane for systemic distribution. As such, transdermal delivery patches as described herein are formulated to deliver dexmedetomidine to the subject through one or more of the subcutis, dermis and epidermis, including the stratum corneum, stratum germinativum, stratum spinosum and stratum basale. Accordingly, a transdermal delivery patch may be applied at any convenient location, such as, for example, the arms, legs, thighs, hips, buttocks, abdomen, back, neck, scrotum, vagina, face, forehead, and behind the ear.

As used herein, “treating” or “treatment” refers to a suppression or an amelioration of the symptoms associated with the condition afflicting the subject, where suppression and amelioration are used in a broad sense to refer to at least a reduction in the magnitude of a parameter, e.g., symptom, associated with the condition being treated, such as post-operative pain. As such, treatment also includes situations where the condition is completely inhibited, e.g., prevented from happening, or stopped, e.g., terminated, such that the subject no longer experiences the condition. As such, treatment includes both preventing and managing a condition.

As used herein, “major neurocognitive disorder” (MND) refers to a type of brain disorder characterized by a significant decline in at least one of the domains of cognition which include executive function, complex attention, language, learning, memory, perceptual-motor, or social cognition. The decline represents a change from a patient's prior level of cognitive ability, is persistent and progressive over time, and is not associated exclusively with an episode of delirium. In addition to the cognitive decline, there must also be a decline in the patient's ability to function and perform everyday tasks. The everyday function of a patient is often evaluated in terms of the ability to perform IADLs (Instrumental Activities of Daily Living), such as managing finances or medications, or, if more severe, ADLs (Activities of Daily Living), such as grooming or feeding oneself. It is often a progressive disorder, and individuals often do not have insight into their deficits.

MND was previously referred to as “Dementia”, but the definition was updated in the DSM-5 because of its limitations, i.e., its common association exclusively with older patients, and that it is often used synonymously with Alzheimer disease. However, MND can affect younger individuals and does not always imply Alzheimer disease as the etiology of cognitive decline.

As used herein, the term “agitation” refers to irritability, emotional outburst, impaired thinking, or excess motor and verbal activity that may occur due to either dysfunction of specific brain regions such as frontal lobes or due to dysfunction of neurotransmitter systems such as dopamine and nor-epinephrine. In the present disclosure, agitation also includes aggression and hyper-arousal, resistant behavior and restlessness. The agitation may be acute, chronic, or episodic.

As used herein, the term “an effective amount” is interchangeable with “therapeutically effective dose,” or “therapeutically effective amount,” and refers to an amount sufficient to produce the desired effect. An effective amount is sufficient to cause an improvement in a condition (e.g., agitation) of the subject.

As used herein, the term “signs of agitation” includes excessive motor activity (e.g., pacing, rocking, gesturing, pointing fingers, restlessness, performing repetitious mannerisms), verbal aggression (e.g., yelling, speaking in an excessively loud voice, using profanity, screaming, shouting, threatening other people), physical aggression (e.g., grabbing, shoving, pushing, clenching hands into fists, resisting, hitting others, kicking objects or people, scratching, biting, throwing objects, hitting self, slamming doors, tearing things, destroying property), and resisting care (e.g., refusing care, constant demands for attention, stubbornness, not following directions).

As used herein, the term “agitation episode” refers to an acute occurrence of agitation, typically characterized by signs of agitation, e.g., agitated speech; restlessness; harming self, others or objects; or resisting care.

As used herein, the term “not agitated” refers to the absence of acute signs of agitation as described herein.

As used herein, the terms “predisposition to agitation” and “predisposed to agitation” refer to a combination of genetic, environmental, and developmental factors that can influence behavior and mental health of a subject. In the context of present disclosure, “predisposition to agitation” refers to a genetic or psychological inclination towards certain behaviors, characterized by signs of agitation.

As used herein, the term “history of agitation” refers to previous documented occurrences of signs of agitation or agitation episodes. Often individuals diagnosed with certain medical disorders, e.g., MND, traumatic brain injury, delirium, schizophrenia, bipolar disorder, have histories of agitation.

As used herein, the term “reduced” refers to a reduction level as compared to a control. For example, in the context of agitation, the skilled artisan will readily understand that the reduction can be measured in terms of well-known agitation scales, e.g., the Agitated Behavior Scale (ABS) the Cohen-Mansfield Agitation Inventory (CMAI), Neuropsychiatric Inventory-Nursing Home Version (NPI-NH), Episodic Agitation Scale-Nursing Home Version (EAS-NH), and the PEC score (Excited component derived from the Positive and Negative Syndrome Scale).

As used herein, the term “adverse reaction” (AE) is any AE caused by a drug or device. Adverse reactions are a subset of all suspected adverse reactions for which there is reason to conclude that the drug or device caused the event. A suspected adverse reaction is any AE for which there is a reasonable possibility that the drug caused the AE. For the purposes of safety reporting to the IND application, “reasonable possibility” means there is evidence to suggest a causal relationship between the drug and the AE. A suspected adverse reaction implies a lesser degree of certainty about causality than an adverse reaction. An AE or suspected adverse reaction is considered unexpected if it is not listed in the investigator brochure or is not listed at the specificity or severity that has been observed; or, if an investigator brochure is not required or available, is not consistent with the risk information described in the general investigational plan or elsewhere in the current application, as amended. Unexpected, as used in this definition, also refers to AEs or suspected adverse reactions that are mentioned in the investigator brochure as occurring with a class of drugs or as anticipated from the pharmacological properties of the drug but are not specifically mentioned as occurring with the particular drug under investigation.

As used herein, the term “flux” refers to the rate of penetration of an active agent through the skin or mucous membrane of the subject following transdermal administration. In some instances, the flux of the dexmedetomidine can be determined by the equation:

$${J}_{skin flux} = P \times C ,$$

where J is the skin flux, C is the concentration gradient across the skin or mucous membrane and P is the permeability coefficient. Skin flux is the change in cumulative amount of drug entering the body across the skin or mucous membrane with respect to time.

### 4.2 Methods of Treating Agitation

In one aspect, the present disclosure provides a method of treating agitation in a subject.

In certain embodiments, the agitation is associated with major neurocognitive disorder (MND) in the subject. MND refers to a type of brain disorder characterized by a significant decline in at least one of the domains of cognition which include executive function, complex attention, language, learning, memory, perceptual-motor, or social cognition. In certain embodiments, the MND is selected from neurocognitive disorder (NCD) due to Alzheimer's disease, vascular NCD, NCD with Lewy bodies, NCD due to Parkinson's, frontotemporal NCD, NCD due to traumatic brain injury, NCD due to HIV infection, substance/medication-induced NCD, NCD due to Huntington's disease, NCD due to prion disease, NCD due to medical condition, NCD due to multiple etiologies, and NCD due to unknown etiology.

In certain embodiments, the MND is NCD due to Alzheimer's disease. In certain embodiments, the MND is dementia, such as, for example, dementia of the Alzheimer's type. In certain embodiments, the MND is NCD due to Alzheimer's disease. In certain embodiments, the MND is dementia (e.g., dementia of the Alzheimer's type). A diagnosis of dementia of probable Alzheimer's Disease type can be diagnosed by those of skill in the art, e.g., based on National Institute on Aging and the Alzheimer's Association (NIA-AA) criteria (2018). In certain embodiments, the subject has a diagnosis of dementia of probable Alzheimer's Disease.

In certain embodiments, the subject is predisposed to agitation. In certain embodiments, the subject has a history of agitation.

In certain embodiments, the subject is not hospitalized. In certain embodiments, the subject is hospitalized.

In certain embodiments, the subject resides in a care facility. In certain embodiments, the care facility is a retirement community. In certain embodiments, the care facility is a medical foster care. In certain embodiments, the care facility is a nursing home. In certain embodiments, the care facility is a memory care unit. In certain embodiments, the care facility is an adult family home.

In certain embodiments, the subject is at least 5 years old, such as, for example, at least 10 years old, at least 15 years old, at least 20 years old, at least 25 years old, at least 30 years old, at least 35 years old, at least 40 years old, at least 45 years old, at least 50 years old, at least 55 years old, at least 60 years old, at least 65 years old, at least 70 years old, at least 75 years old, at least 80 years old, or at least 85 years old. In certain embodiments, the subject has a body weight of greater than 50 kg, such as, for example, greater than 55 kg, greater than 60 kg, greater than 65 kg, greater than 70 kg, greater than 75 kg, or greater than 80 kg.

In certain embodiments, the subject had two or more episodes of agitation over a 7-day lookback period prior to applying the one or more transdermal patches, e.g., three or more, four or more, five or more, six or more, seven or more, eight or more, nine or more, or ten or more episodes of agitation. An episode of agitation, as used herein, may impair social activities, may require medical intervention, or may impair ability for functional activities of daily living.

In certain embodiments, the subject had two or more episodes of agitation over a 14-day lookback period prior to applying the one or more transdermal patches, e.g., three or more, four or more, five or more, six or more, seven or more, eight or more, nine or more, or ten or more episodes of agitation. An episode of agitation, as used herein, may impair social activities, may require medical intervention, or may impair ability for functional activities of daily living.

In certain embodiments, the subject has an ABS score of at least 22 at least once when assessed during the four days prior to application of the one or more transdermal delivery patches.

In certain embodiments, an episode of agitation may impair social activities, wherein the subject shows signs of verbal aggression (e.g., yelling, speaking in an excessively loud voice, using profanity, screaming, shouting, and/or threatening other people). In certain embodiments, an episode of agitation may require medical intervention, e.g., if the subject shows signs of physical aggression (e.g., grabbing, shoving, pushing, clenching hands into fists, resisting, hitting others, kicking objects or people, scratching, biting, throwing objects, hitting self, slamming doors, tearing things), and destroying property. In certain embodiments, an episode of agitation may impair a subject's ability to carry out functional activities of daily living, wherein the subject shows signs of excessive motor activity (e.g., pacing, rocking, gesturing, pointing fingers, restlessness, performing repetitious mannerisms). In certain embodiments, an episode of agitation may disrupt delivery of medical care and/or functional activities of daily living, wherein the subject is highly resistant (e.g., refusing care, being difficulty, repetitively demanding attention, not following directions).

In some embodiments, the subject has an Estimated Glomerular Filtration Rate (eGFR) above the lower limit of the age- and gender-specific range as provided in Table 11.

In certain embodiments, the method of treating agitation comprises applying one or more transdermal delivery patches to a skin surface of a subject that is not agitated at the time of application. In certain embodiments, the method of treating agitation comprises applying one or more transdermal delivery patches to a skin surface of a subject that is not exhibiting signs of acute agitation. The transdermal delivery patch comprises a pharmaceutical composition comprising dexmedetomidine. In certain embodiments, the patches are maintained on the skin surface of the patient for at least 72 hours, e.g., at least 84 hours, at least 96 hours, or up to 120 hours. In certain embodiments, the one or more transdermal delivery patches deliver a therapeutically effective non-sedating amount of dexmedetomidine to the subject sufficient to treat agitation in the subject.

In certain embodiments, the one or more transdermal delivery patches are self-applied by the subject. In certain embodiments, the one or more transdermal delivery patches are applied by someone other than the subject, e.g., a caregiver, nurse, or physician.

The one or more transdermal delivery patches are applied to the skin surface of a subject. In certain embodiments, the skin surface is not within manual reach of the subject.

In certain embodiments, the skin surface is selected from the arms, legs, thighs, hips, buttocks, abdomen, back, neck, scrotum, vagina, face, forehead, and behind the ear. In certain embodiments, the skin surface is the upper back or lower back of the subject.

In certain embodiments, the one or more transdermal delivery patches are maintained on the skin surface of the subject for a duration at least 72 hours, e.g., at least 80 hours, at least 90 hours, at least 96 hours, or at least 100 hours. “Maintained,” as used herein, means that at least 90% of the one or more transdermal delivery patches remains in contact with the skin surface of the subject. For example, the one or more transdermal delivery patches are not removed or otherwise tampered with so as to prevent at least 90% contact between the one or more transdermal delivery patches and the skin surface. In certain embodiments, for proper maintenance, subjects avoid activities that may lead to excessive sweating while the one or more transdermal delivery patches are applied to the skin surface. In certain embodiments, subjects may take up to 1 shower daily.

In certain embodiments, the one or more transdermal delivery patches are maintained on the skin surface of the subject from 72 to 100 hours, e.g., from 72 to 96 hours, from 72 to 70 hours, from 72 to 80 hours, from 80 to 100 hours, from 80 to 96 hours, from 80 to 90 hours, from 90 to 100 hours, from 90 to 96 hours, or from 96 to 100 hours. In certain embodiments, the one or more transdermal delivery patches are maintained on the skin surface of the subject for 2 to 6 days, e.g., 2 to 5 days, 2 to 4 days, 2 to 3 days, 3 to 6 days, 3 to 5 days, 3 to 4 days, 4 to 6 days, or 4 to 5 days. In certain embodiments, the one or more transdermal delivery patches are maintained on the skin surface of the subject for 4 days. In certain embodiments, the one or more transdermal delivery patches are maintained on the skin surface of the subject for 96 hours±3 hours.

In certain embodiments, the method further comprises removing the one or more transdermal delivery patches from the skin surface of the subject. In certain embodiments, the one or more transdermal delivery patches are removed by the subject. In certain embodiments, the one or more transdermal delivery patches are removed by someone other than the subject.

In certain embodiments, the method further comprises applying a further one or more transdermal delivery patches to a skin surface of the subject.

In certain embodiments, the subject has an ABS score of >22 at least once after removal of the one or more transdermal delivery patches and before applying the further one or more transdermal delivery patches to a skin surface of the subject.

In certain embodiments, applying the further one or more transdermal delivery patches occurs at least 10 days, such as, for example, at least 11 days, at least 12 days, at least 13 days, at least 14 days, at least 15 days, at least 16 days, at least 17 days, at least 18 days, at least 19 days, at least 20 days, at least 21, at least 22, at least 23, at least 24, at least 25, at least 26, at least 27, at least 28, at least 29, or at least 30, at least 31, at least 32, at least 33, at least 34, at least 35, at least 36, at least 37, at least 38, at least 39, or at least 40 days after the previous applying of one or more transdermal delivery patches to the skin surface of the subject. In certain embodiments, applying the further one or more transdermal delivery patches occurs at least 14 days after the previous applying of one or more transdermal delivery patches to the skin surface of the subject. In certain embodiments, applying the further one or more transdermal delivery patches occurs 14 days after the previous applying of one or more transdermal delivery patches to the skin surface of the subject.

In certain embodiments, the further one or more transdermal delivery patches are maintained on the skin surface of the subject for a duration at least 72 hours, such as, for example, at least 84 hours, at least 96 hours, at least 108 hours, at least 120 hours, or at least 132 hours. In certain embodiments, the further one or more transdermal delivery patches are maintained on the skin surface of the subject for a duration at least 96 hours. In certain embodiments, the further one or more transdermal delivery patches are maintained on the skin surface of the subject for 84 to 108 hours, e.g., from 90 to 100 hours. In certain embodiments, the further one or more transdermal delivery patches are maintained on the skin surface of the subject for 96 hours. In certain embodiments, the further one or more transdermal delivery patches are maintained on the skin surface of the subject for 4 days. In certain embodiments, the further one or more transdermal delivery patches are maintained on the skin surface of the subject for 96 hours±3 hours.

In certain embodiments, the method further comprises removing the further one or more, such as, for example, two or more, three or more, four or more, or five or more transdermal delivery patches from the skin surface of the subject.

In certain embodiments, a Folstein Mini-Mental State Exam (MMSE) is used to assess the subject's cognitive function, to characterize the severity of Alzheimer's disease, and to assess changes in cognition associated with treatment. The Folstein MMSE is a formal mental status examination frequently used to assess cognition. Domains measured by the MMSE include orientation to time and place, registration, attention and calculation, recall, naming, repetition, comprehension, reading, writing, and drawing. The maximum of total points on this test is 30. In certain embodiments, the subject has a MMSE score of greater than 20, such as, for example, greater than 21, greater than 22, greater than 23, greater than 24, or greater than 25, when administered on one of the 7 days prior to applying the one or more transdermal patches. In certain embodiments, the subject has a MMSE score of greater than 23, when administered on one of the 7 days prior to applying the one or more transdermal patches. In certain embodiments, the MMSE can be administered on one of the 7 days prior to applying the one or more transdermal delivery patches and again at end of study. In certain embodiments, the subject's MMSE score improves following treatment with the one or more transdermal delivery patches.

In certain embodiments, a John Hopkins Fall Risk Assessment Score is used to assess the subject's cognitive function, to characterize the severity of Alzheimer's disease, and to assess changes in cognition associated with treatment. In certain embodiments, the subject exhibits a John Hopkins Fall Risk Assessment Score of greater than 10, e.g., greater than 11, greater than 12, greater than 13, greater than 14, or greater than 15.

In certain embodiments, the frequency or severity of agitation in the subject is reduced following application of the one or more transdermal delivery patches or the further one or more transdermal delivery patches to a skin surface and maintenance on the skin surface as described herein. In certain embodiments, the frequency or severity of agitation in the subject is reduced following application of the one or more transdermal delivery patches or the further one or more transdermal delivery patches to a skin surface and maintenance on the skin surface as described herein.

In certain embodiments, reduction in frequency or severity of agitation behaviors is determined by a change in one or more of the following scores:
Agitated Behavior Scale (ABS),
Cohen Mansfeld Agitation Inventory (CMAI),
Clinical Global Impression (Severity CGI-S); and Change (CGI-C),
Neuropsychiatric Inventory-Nursing Home Version (NPI-NH), and
Episodic Agitation Scale-Nursing Home (EAS-NH).

In certain embodiments, the Agitated Behavior Scale (ABS) is used to assess agitated behavior. ABS is particularly useful in serial assessments for the evaluation of interventions to reduce agitation. The ABS is a 14-item scale developed to allow objective assessment of agitated behavior, particularly serial assessments for the evaluation of interventions to reduce agitation. The ABS items are as follows: short attention span, easy distractibility, inability to concentrate, impulsive, impatient, low tolerance for pain or frustration, uncooperative, resistant to care or demanding, violent and or threatening violence toward people or property, explosive or unpredictable anger, rocking, rubbing, moaning or other self-stimulating behavior, pulling at tubes or restraints, wandering from treatment areas, restlessness, pacing, or excessive movement, repetitive behaviors (motor and/or verbal), rapid, loud or excessive talking, sudden changes of mood, easily initiated or excessive crying and/or laughing, and self-abusiveness (physical and/or verbal).

Behavior is rated using the following scale:
1. Absent: the behavior is not present.
2. Present to a Slight Degree: the behavior is present but does not prevent the conduct of other, contextually appropriate behavior (the individual may redirect spontaneously, or the continuation of the agitated behavior does not disrupt appropriate behavior).
3. Present to a Moderate Degree: the individual needs to be redirected from an agitated state to an appropriate behavior but is able to benefit from such cueing.
4. Present to an Extreme Degree: the individual is not able to engage in appropriate behavior due to the interference of the agitated behavior, even when external cueing or redirection is provided.

The ABS Total Score ranges from 14 to 56. A total score of 21 or less is categorized as normal; 22 to 28 is categorized as mild; 29 to 35 is categorized as moderate; and 36 or more is categorized as severe agitation.

In certain embodiments, treatment with the one or more transdermal delivery patches changes (i.e., improves) the subject's ABS categorization from severe to moderate, from severe to mild, or from severe to normal. In certain embodiments, treatment with the one or more transdermal delivery patches decreases the subject's ABS score from severe to moderate, from moderate to mild, or from mild to normal. In certain embodiments, treatment with the one or more transdermal delivery patches decreases the subject's ABS score from severe to mild, severe to normal, or from moderate to normal.

In certain embodiments, the reduction in frequency or severity of agitation is determined by a change in ABS score over time following treatment with the one or more transdermal delivery patches compared to a baseline ABS score prior to application of the one or more transdermal delivery patches. In certain embodiments, the subject's baseline ABS score is the mean score of daily ABS measurements for the four days prior to application of the one or more transdermal delivery patches (i.e., day-4 to day-1). In certain embodiments, the subject has an ABS score of at least 22 at least once when assessed during the four days prior to application of the one or more transdermal delivery patches. In certain embodiments, the subject has an ABS score of at least 22 at least once when assessed during the four days prior to application of the further one or more transdermal delivery patches. In certain embodiments, the subject's ABS score is measured daily from 1-14 days following application and maintenance of the one or more transdermal delivery patches with the skin surface of the subject. In certain embodiments, the subject's ABS score is assessed 96 hours following application and maintenance of the one or more transdermal delivery patches with the skin surface of the subject (e.g., the morning of day 5 of the treatment period).

In certain embodiments, the subject's ABS score 96 hours following application and maintenance of the one or more transdermal delivery patches with the skin surface is reduced compared to the subject's baseline ABS score.

In certain embodiments, the subject's ABS score 168 hours following application and maintenance of the one or more transdermal delivery patches with the skin surface for four days is reduced compared to the subject's baseline ABS score.

In certain embodiments, NPI-NH is used to assess dementia related behavioral symptoms including Alzheimer's disease. NPI-NH assesses the severity and frequency of each neuropsychiatric symptom and the amount of caregiver/study partner distress engendered by each of the neuropsychiatric symptoms based on a series of scripted questions administered to the patient's caregiver. The scale is administered by a certified clinician with the patient's caregiver serving as the informant. Based upon a positive response to the screening question, follow-up questions are read, and yes/no responses are recorded. Both frequency and severity are evaluated based upon the most abnormal behavior revealed in the follow-up questions. After frequency and severity have been determined, the informant is asked to rate the level of disruptiveness of the behavior. After frequency and severity have been determined, the caregiver is asked to rate the level of disruptiveness of the behavior.

In certain embodiments, the reduction in frequency or severity of agitation is determined by a reduction in NPI-NH score over time following treatment with the one or more transdermal delivery patches compared to a baseline NPI-NH score measured prior to application of the one or more transdermal delivery patches. In certain embodiments, the subject's baseline NPI-NH score is assessed on the same day, and prior to, application of the one or more transdermal delivery patches to the skin surface of the subject, e.g., 30 minutes to 12 hours, 1 hour, 2 hours, 3 hours, 4 hours, 5 hours, 6 hours, 7 hours, 8 hours, 9 hours, 10 hours, or 11 hours, prior to application of the one or more transdermal delivery patches. In certain embodiments, the subject's NPI-NH score is assessed after application and maintenance (e.g., at least 4 days, such as from 4 to 9 days, including for 5, 6, 7, 8, or 9 days, in particular for 7 days after application and maintenance) of the one or more transdermal delivery patches to the skin surface of the subject for 4 days.

In certain embodiments, the subject's NPI-NH is assessed with a 7-day lookback period (e.g., day-7 to day-1 of the treatment period for a baseline NPI-NH or day 1 to day 7 for a treatment NPI-NH).

### Frequency:

1. Rarely—less than once per week
2. Sometimes—about once per week
3. Often—several times per week but less than every day
4. Very Often—once or more per day

### Severity:

1. Mild—behavior is stressful to the resident but can be controlled
2. Moderate—behaviors are stressful for and are upsetting to the resident and are difficult to control
3. Severe—agitation is very stressful and upsetting to the resident and is very difficult or impossible to control. There is a possibility they may injure themselves and medications are often required.

### Occupational Disruptiveness:

0. Not at all
1. Minimally (almost no change in work routine)
2. Mildly (some change in work routine but little time rebudgeting required)
3. Moderately (disrupts work routine, requires time rebudgeting)
4. Severely (disruptive, upsetting to staff and other residents, major time infringement)
5. Very severely or extremely (very disruptive, major source of distress for staff and other residents, requires time usually devoted to other residents or activities).

In certain embodiments, the subject's NPI-NH score 168 hours following application of the one or more transdermal delivery patches to the skin surface of the subject and maintenance for 4 days (i.e., on day 8 of the treatment period) is reduced compared to the subject's baseline NPI-NH score. In certain embodiments, the subject's NPI-NH score 168 hours (following application of the one or more transdermal delivery patches to the skin surface of the subject and maintenance for 4 days (i.e., on day 8 of the treatment period) is reduced compared to the subject's baseline NPI-NH score, wherein both NPI-NH scores are assessed with a 7-day lookback period.

In certain embodiments, the Cohen-Mansfeld Agitation Inventory (CMAI) is used to assess agitation. The CMAI is a 29-item scale designed to systematically assess agitation. Institutionalized older adults are rated regarding the frequency with which they manifest physically aggressive, physically non-aggressive and verbally agitated behaviors. The CMAI is applied using a 14-day lookback, rating the frequency of occurrence of each item over this 14-day interval on a 7-point scale as follows:
1. Never
2. Less than once a week
3. Once or twice a week
4. Several times a week
5. Once or twice a day
6. Several times a day
7. Several times an hour
   The CMAI is performed via a formal interview with an informant by a certified rater. The informant will be a staff member of the facility or site who is in contact with the subject for a combined average of at least 2 hours a day for a minimum of 3 days/week and has had direct observation of the subject's behavior.

In certain embodiments, the CMAI baseline score is based on a lookback period of 14 days prior to application of the one or more transdermal delivery patches to the skin surface of the subject. In certain embodiments the CMAI follow-up score will be obtained 15 days after application of one or more transdermal delivery patches to the skin surface of the subject. In some embodiments, the CMAI total score 15 days after application of the one or more transdermal delivery patches with the skin surface of the subject for 4 days (i.e., 96 hours) is reduced compared to the subject's baseline CMAI total score.

In certain embodiments, the EAS-NH is used to assess the frequency and severity of agitation on a daily basis. In certain embodiments, the reduction in frequency or severity of agitation is determined by a reduction in EAS-NH score over time. In certain embodiments, an initial EAS-NH score is assessed on a daily basis for each 24 hour period of the 7 days prior to application of the one or more transdermal delivery patches. In certain embodiments, a subsequent EAS-NH score is assessed for each 24 hour period after application of the one or more transdermal delivery patches to the skin surface of the subject, for example, 24 hours, 48 hours, 72 hours, 96 hours, 120 hours, 168 hours, 192 hours, 216 hours, 240 hours, 264 hours, 288 hours, or 336 hours after application.

In certain embodiments, the subject's EAS-NH score at one or more of the following timepoints following application and maintenance of the one or more transdermal delivery patches with the skin surface of the subject for 4 days (i.e., 96 hours) is reduced compared to the subject's baseline EAS-NH score assessed prior to application of the one or more transdermal delivery patches: 24 hours, 48 hours, 72 hours, 96 hours, 120 hours, 168 hours, 192 hours, 216 hours, 240 hours, 264 hours, 288 hours, or 336 hours after application.

In certain embodiments, the Clinical Global Impression-Severity (CGI-S) is used to assess the clinician's impression of the severity of the subject's agitation. The Clinical Global Impression-Severity is the rating of clinical severity based on the clinician's judgment. The lookback period for the CGI-S scores obtained at screening are based on the 4-day and 7-day periods prior to application of the one or more transdermal delivery patch. The CGI-S is a 7-point (1-7) clinician-rated scale in which higher ratings indicate greater severity of agitation. In certain embodiments, the subject's CGI-S score is reduced following treatment with the one or more transdermal delivery patches compared to a baseline CGI-S score measured prior to application of the one or more transdermal delivery patches. In certain embodiments, the subject's baseline CGI-S score is assessed on the same day, but prior to, application of the one or more transdermal delivery patches to the skin surface of the subject. In certain embodiments, the subject's CGI-S score is assessed 96 hours, 120 hours, 144 hours, and/or 168 hours following application of the one or more transdermal delivery patches, wherein the one or more transdermal delivery patches are maintained on the skin surface of the subject for 4 days.

In certain embodiments, the Clinical Global Impression-Change (CGI-C) is used to assess the clinician's impression of change in the subject's agitation. The CGI=C is a rating of the change in level of agitation based on the clinician's judgment. The CGI-C score is derived from a 7-point (1-7) rating scale in which the change in agitation (improvement or worsening) is rated as no change, mild, moderate, or much change. In certain embodiments, the CGI-C score shows improvement following treatment with the one or more transdermal delivery patches (for example a rating of mildly, moderately, or much improved compared to the specified lookback period). In certain embodiments, the CGI-C score is assessed 96 hours, 120 hours, 144 hours, and/or 168 hours following application of the one or more transdermal delivery patches, wherein the one or more transdermal delivery patches are maintained on the skin surface of the subject for 4 days.

In certain embodiments, the one or more transdermal delivery patches deliver a therapeutically effective non-sedating amount of dexmedetomidine to the subject for at least two days, at least three days, or at least four days. As used herein, “non-sedative” refers to an amount of dexmedetomidine that does not cause complete sedation of the subject. Suitable protocols for determining level of sedation may include, but are not limited to, the Ramsay Sedation Scale, the Vancouver Sedative Recovery Scale, the Glasgow Coma Scale modified by Cook and Palma, the Comfort Scale, the New Sheffield Sedation Scale, the Sedation-Agitation Scale, and the Motor Activity Assessment Scale, and the Wilson Sedation Score. In certain embodiments, the level of sedation is evaluated using the Wilson Sedation Scale.

In certain embodiments, the one or more transdermal delivery patches delivers a non-sedative amount of dexmedetomidine to the subject as measured by, e.g., the Wilson Sedation Score. In certain embodiments, the subject's Wilson Sedation Score does not exceed 3 post-operatively while the transdermal delivery patch is adhered to the skin surface of the subject, e.g., for at least 96 hours post-operatively. In certain embodiments, the subject's Wilson Sedation Score is less than 3, for example, 2 or 1. In certain embodiments, the subject's Wilson Sedation Score is 1. In certain embodiments, the one or more transdermal delivery patches or transdermal delivery patch system delivers a non-sedative amount of dexmedetomidine to the subject.

The flux of an active agent by transdermal administration is the rate of penetration of the active agent through the skin or mucous membrane of the subject. In some instances, the flux of the dexmedetomidine can be determined by the equation:

$${J}_{skin flux} = P \times C$$

where J is the skin flux,
C is the concentration gradient across the skin or mucous membrane; and
P is the permeability coefficient.

Skin flux is the change in cumulative amount of drug that crosses a unit area (i.e., square cm) of the skin, other suitable skin source (e.g., skin obtained during abdominoplasty procedure), or mucous membrane with respect to time.

The transdermal dexmedetomidine flux may be determined using any convenient protocol, e.g., protocols employing human cadaver skin with epidermal layers (stratum corneum and viable epidermis) in a Franz cell having donor and receptor sides clamped together and receptor solution containing phosphate buffer. In one exemplary protocol, human cadaver skin is used, and epidermal layers (stratum corneum and viable epidermis) are separated from the full-thickness skin as skin membrane. Samples are die-cut with an arch punch to a final diameter of about 2.0 cm². The release liner of the transdermal delivery patch is removed, and the adhesive surface is adhered to the top of the epidermis/stratum corneum with the dexmedetomidine adhesive layer facing the outer surface of the stratum corneum. Gentle pressure is applied to effect good contact between the adhesive layer and stratum corneum. The donor and receptor sides of the Franz cell are clamped together and the receptor solution containing a phosphate buffer at pH 6.5 and 0.01% gentamicin is added to the Franz cell. The cells are kept at 32° C.-35° C. for the duration of the experiment. Samples of the receptor solution are taken at regular intervals and the active agent concentration is measured by HPLC. The removed receptor solution is replaced with fresh solution to maintain sink conditions. The flux is calculated from the slope of cumulative amount of the drug permeated into the receiver compartment versus time plot.

In certain embodiments, the one or more transdermal delivery patches provide an in vitro average flux of dexmedetomidine of 0.005 to 5 μg/cm²/hr at any time after application of the one or more patches to a skin surface and maintenance of contact between the skin surface and the one or more patch (e.g., for 24 hours, 48 hours, 72 hours, 96 hours, or 120 hours after application).

In certain embodiments, the one or more transdermal delivery patches provide an in vitro average flux of dexmedetomidine of 0.5 μg/cm²/hr to 3 μg/cm²/hr at any time after application of the one or more patch and maintenance of contact between the skin surface and the one or more patches (e.g., for 24 hours, 48 hours, 72 hours, 96 hours, and/or 120 hours after application), e.g., from 0.5 g/cm²/hr to 2 μg/cm²/hr, or from 0.5 μg/cm²/hr to 1 μg/cm²/hr.

In certain embodiments, the one or more transdermal delivery patches provide an in vitro average flux of dexmedetomidine of 0.5 μg/cm²/hr to 3 μg/cm²/hr 24 hours after application of the one or more transdermal delivery patches and maintenance of contact between the skin surface and the one or more transdermal delivery patches, e.g., from 0.5 g/cm²/hr to 2 μg/cm²/hr, or from 0.5 μg/cm²/hr to 1 μg/cm²/hr.

In certain embodiments, the one or more transdermal delivery patches provide an in vitro average flux of dexmedetomidine of 0.5 μg/cm²/hr to 3 μg/cm²/hr 48 hours after application of the one or more transdermal delivery patches and maintenance of contact between the skin surface and the one or more transdermal delivery patches, e.g., from 0.5 μg/cm²/hr to 2 μg/cm²/hr, or 0.5 μg/cm²/hr to 1 μg/cm²/hr.

In certain embodiments, the one or more transdermal delivery patches provide an in vitro average flux of dexmedetomidine of 0.5 g/cm²/hr to 3 μg/cm²/hr 72 hours after application of the one or more transdermal delivery patches and maintenance of contact between the skin surface and the one or more transdermal delivery patches, e.g., from 0.5 μg/cm²/hr to 2 μg/cm²/hr, or 0.5 μg/cm²/hr to 1 μg/cm²/hr.

In certain embodiments, the one or more transdermal delivery patches provide an in vitro average flux of dexmedetomidine of 0.5 μg/cm²/hr to 3 μg/cm²/hr 96 hours after application of the one or more transdermal delivery patches and maintenance of contact between the skin surface and the one or more transdermal delivery patches, e.g., from 0.5 μg/cm²/hr to 2 μg/cm²/hr, or from 0.5 μg/cm²/hr to 1 μg/cm²/hr.

In certain embodiments, the one or more transdermal delivery patches provide an in vitro average flux of dexmedetomidine of 0.5 μg/cm²/hr to 3 μg/cm²/hr 120 hours after application of the one or more transdermal delivery patches and maintenance of contact between the skin surface and the one or more transdermal delivery patches, e.g., from 0.5 μg/cm²/hr to 2 μg/cm²/hr, or from 0.5 μg/cm²/hr to 1 μg/cm²/hr.

In certain embodiments, the one or more transdermal delivery patches provide an in vitro dexmedetomidine peak flux of 0.1 μg/cm²/hr to 3 μg/cm²/hr, e.g., from 0.1 μg/cm²/hr to 2.5 g/cm²/hr, from 0.1 μg/cm²/hr to 2.0 μg/cm²/hr, from 0.1 μg/cm²/hr to 1.5 μg/cm²/hr, from 0.1 μg/cm²/hr to 1 μg/cm²/hr, or from 0.1 μg/cm²/hr to 0.8 g/cm²/hr.

In certain embodiments, the one or more transdermal delivery patches provide an in vitro dexmedetomidine peak flux of 0.5 to 3 μg/cm²/hr, e.g., from 0.5 μg/cm²/hr to 2.5 μg/cm²/hr, from 0.5 μg/cm²/hr to 2.0 μg/cm²/hr, from 0.5 μg/cm²/hr to 1.5 μg/cm²/hr, or from 0.5 μg/cm²/hr to 1 μg/cm²/hr.

In certain embodiments, peak dexmedetomidine flux is reached 2 hours or more after applying the one or more transdermal delivery patches, e.g., 4 hours or more, 6 hours or more, 12 hours or more, 18 hours or more, or 24 hours or more after applying the one or more transdermal delivery patches and maintenance of contact between the skin surface and the one or more transdermal delivery patches.

In embodiments where the one or more transdermal delivery patches include a release liner covering the adhesive surface, the method further comprises removing the release liner to expose the adhesive surface prior to applying the one or more transdermal delivery patches to the skin surface of the subject.

In certain embodiments, the target dosage is an amount that when applied to a subject provides for a systemic amount of dexmedetomidine that gives a desired mean plasma concentration of dexmedetomidine at specific times. In other embodiments, the target dosage is an amount that when applied to a subject provides for a steady state mean plasma concentration of the dexmedetomidine throughout a dosage interval or management protocol. In other embodiments, the target dosage is an amount that when applied to a subject provides for a particular rate of delivery of dexmedetomidine to the subject in vivo.

In certain embodiments, applying and maintaining the one or more transdermal delivery patches delivers a target amount of dexmedetomidine, such as, for example, an average cumulative amount of dexmedetomidine delivered over the course of a dosage interval (e.g., 4 days or longer). The term “target cumulative amount” is meant the total quantity of dexmedetomidine that is delivered to the subject through the skin and may vary due to skin or mucous membrane permeability and metabolic activity of the site of application. In certain embodiments, the average cumulative amount of dexmedetomidine may be 5 μg/cm² or greater over a dosage interval (e.g., 4 days or longer), e.g., 25 μg/cm² or greater, 50 μg/cm² or greater, 75 μg/cm² or greater, 100 μg/cm² or greater, 125 μg/cm² or greater, 200 μg/cm² or greater, or 300 μg/cm² or greater over the dosage interval. In certain embodiments, average cumulative amount of dexmedetomidine delivery over a dosage interval ranges such as from 5 μg/cm² to 500 μg/cm², such as from 25 μg/cm² to 400 μg/cm², or from 50 μg/cm² to 350 μg/cm². In certain embodiments, the average cumulative amount of dexmedetomidine may be 200 μg/cm² or greater over a dosage interval (e.g., 3 days or longer), e.g., 225 μg/cm² or greater, 250 μg/cm² or greater, 275 μg/cm² or greater, 300 μg/cm² or greater, 325 μg/cm² or greater over the dosage interval. In certain embodiments, the average cumulative amount of dexmedetomidine delivery over a dosage interval is from 250 μg/cm² to 350 μg/cm², from 275 μg/cm² to 350 μg/cm², from 300 μg/cm² to 350 μg/cm², from 325 μg/cm² to 350 μg/cm², from 250 μg/cm² to 325 μg/cm², from 275 μg/cm² to 325 μg/cm², from 300 μg/cm² to 325 μg/cm², from 250 μg/cm² to 300 μg/cm², from 275 μg/cm² to 300 μg/cm², or from 250 μg/cm² to 300 μg/cm².

### 4.3 Transdermal Delivery Patches

The methods herein utilize one or more transdermal delivery patches that provide extended release of the dexmedetomidine formulated within the patch. The transdermal delivery patches described herein, in certain embodiments, release dexmedetomidine over a period of at least 4 days.

In certain embodiments, the dexmedetomidine is a pharmaceutically acceptable salt of dexmedetomidine. In certain embodiments, the dexmedetomidine is dexmedetomidine hydrochloride.

In certain embodiments, the one or more transdermal delivery patches comprise from 1 to 4 mg dexmedetomidine. In certain embodiments, the one or more transdermal delivery patches comprise from 1 mg to 3.5 mg dexmedetomidine, such as, for example, from 1 mg to 2.75 mg, from 1 mg to 2.5 mg, from 1 mg to 2.25 mg, from 1 mg to 2 mg, from 1 mg to 1.75 mg, from 1 mg to 1.5 mg, from 1 mg to 1.25 mg, from 1.25 mg to 3.5 mg, from 1.25 mg to 3 mg, from 1.25 mg to 2.75 mg, from 1.25 mg to 2.5 mg, from 1.25 mg to 2.25 mg, from 1.25 mg to 2 mg, from 1.25 mg to 1.75 mg, from 1.25 mg to 1.5 mg, from 1.5 mg to 3.5 mg, from 1.5 mg to 3 mg, from 1.5 mg to 2.75 mg, from 1.5 mg to 2.5 mg, from 1.5 mg to 2.25 mg, from 1.5 mg to 2 mg, from 1.5 mg to 1.75 mg, from 1.75 mg to 3.5 mg, from 1.75 mg to 3 mg, from 1.75 mg to 2.75 mg, from 1.75 mg to 2.5 mg, from 1.75 mg to 2.25, from 1.75 mg to 2 mg, from 2 mg to 3.5 mg, from 2 mg to 3 mg, from 2 mg to 2.75 mg, from 2 mg to 2.5 mg, from 2 mg to 2.25 mg, from 2.25 mg to 3.5 mg, from 2.25 mg to 3 mg, from 2.25 mg to 2.75 mg, from 2.25 mg to 2.5 mg, from 2.5 mg to 3.5 mg, from 2.5 mg to 3 mg, from 2.5 mg to 2.75 mg, from 2.5 mg to 3.5 mg, from 2.5 mg to 3.0 mg, from 2.5 mg to 2.75 mg, from 2.75 mg to 3.5 mg, from 2.75 mg to 3 mg, or from 3 mg to 3.5 mg.

In certain embodiments, the one or more transdermal delivery patches comprise a total of from 1.3 mg to 1.6 mg dexmedetomidine or from 1.4 mg to 1.5 mg dexmedetomidine. In certain embodiments, the one or more transdermal delivery patches comprise a total of from 1.8 mg to 2.3 mg dexmedetomidine or from 2.0 mg to 2.1 mg dexmedetomidine. In certain embodiments, the one or more transdermal delivery patches comprise a total of from 2 mg to 2.4 mg dexmedetomidine or from 2.1 to 2.2 mg dexmedetomidine. In certain embodiments, the one or more transdermal delivery patches comprise a total of from 2.6 mg to 3.2 mg dexmedetomidine or from 2.9 mg to 3 mg dexmedetomidine. In certain embodiments, the one or more transdermal delivery patches comprise a total of from 3 mg to 4 mg dexmedetomidine or from 3.5 mg to 3.7 mg dexmedetomidine.

In certain embodiments, one or more transdermal delivery patches comprise a total of from 1 mg to 2 mg dexmedetomidine, e.g., from 1.3 mg to 1.6 mg dexmedetomidine, or from 1.4 mg to 1.5 mg dexmedetomidine.

In certain embodiments, the transdermal delivery patch comprises a drug layer affixed to a backing layer, wherein the drug layer comprises from 1 mg to 4 mg dexmedetomidine and a pressure sensitive adhesive, and the drug layer has an adhesive surface suitable for adhesion to a skin surface.

In certain embodiments, the drug layer is a single layer comprising dexmedetomidine and a pressure sensitive adhesive. In certain embodiments, the drug layer is affixed to a backing layer, e.g., by lamination. The surface of the drug layer opposite the backing layer is adhesive and adheres to a skin surface of a subject.

In certain embodiments, the drug layer comprises from 1 mg to 4 mg dexmedetomidine. In certain embodiments, the drug layer comprises from 1 mg to 3.5 mg dexmedetomidine, such as, for example, from 1 mg to 2.75 mg, from 1 mg to 2.5 mg, from 1 mg to 2.25 mg, from 1 mg to 2 mg, from 1 mg to 1.75 mg, from 1 mg to 1.5 mg, from 1 mg to 1.25 mg, from 1.25 mg to 3.5 mg, from 1.25 mg to 3 mg, from 1.25 mg to 2.75 mg, from 1.25 mg to 2.5 mg, from 1.25 mg to 2.25 mg, from 1.25 mg to 2 mg, from 1.25 mg to 1.75 mg, from 1.25 mg to 1.5 mg, from 1.5 mg to 3.5 mg, from 1.5 mg to 3 mg, from 1.5 mg to 2.75 mg, from 1.5 mg to 2.5 mg, from 1.5 mg to 2.25 mg, from 1.5 mg to 2 mg, from 1.5 mg to 1.75 mg, from 1.75 mg to 3.5 mg, from 1.75 mg to 3 mg, from 1.75 mg to 2.75 mg, from 1.75 mg to 2.5 mg, from 1.75 mg to 2.25, from 1.75 mg to 2 mg, from 2 mg to 3.5 mg, from 2 mg to 3 mg, from 2 mg to 2.75 mg, from 2 mg to 2.5 mg, from 2 mg to 2.25 mg, from 2.25 mg to 3.5 mg, from 2.25 mg to 3 mg, from 2.25 mg to 2.75 mg, from 2.25 mg to 2.5 mg, from 2.5 mg to 3.5 mg, from 2.5 mg to 3 mg, from 2.5 mg to 2.75 mg, from 2.5 mg to 3.5 mg, from 2.5 mg to 3.0 mg, from 2.5 mg to 2.75 mg, from 2.75 mg to 3.5 mg, from 2.75 mg to 3 mg, or from 3 mg to 3.5 mg.

In certain embodiments, the drug layer comprises from 1.3 mg to 1.6 mg dexmedetomidine or from 1.4 mg to 1.5 mg dexmedetomidine. In certain embodiments, the drug layer comprises from 1.8 mg to 2.3 mg dexmedetomidine or from 2.0 mg to 2.1 mg dexmedetomidine. In certain embodiments, the drug layer comprises from 2 mg to 2.4 mg dexmedetomidine or from 2.1 to 2.2 mg dexmedetomidine. In certain embodiments, the drug layer comprises from 2.6 mg to 3.2 mg dexmedetomidine or from 2.9 mg to 3 mg dexmedetomidine. In certain embodiments, the drug layer comprises from 1 mg to 2 mg dexmedetomidine, e.g., from 1.3 mg to 1.6 mg dexmedetomidine, or from 1.4 mg to 1.5 mg dexmedetomidine.

Pressure sensitive adhesives include, but are not limited to, poly-isobutene adhesives, polyisobutylene adhesives, poly-isobutene/polyisobutylene adhesive mixtures, carboxylated polymers, and acrylic or acrylate copolymers, such as carboxylated acrylate copolymers.

Where the pressure sensitive adhesive includes polybutene, the polybutene may be saturated polybutene. Alternatively, the polybutene may be unsaturated polybutene. Still further, the polybutene may be a mixture or combination of saturated polybutene and unsaturated polybutene. In certain embodiments, the pressure sensitive adhesive may include a composition that is, or is substantially the same as, the composition of Indopol® L-2, Indopol® L-3, Indopol® L-6, Indopol® L-8, Indopol® L-14, Indopol® H-7, Indopol® H-8, Indopol® H-15, Indopol® H-25, Indopol® H-35, Indopol® H-50, Indopol® H-100, Indopol® H-300, Indopol® H-1200, Indopol® H-1500, Indopol® H-1900, Indopol® H-2100, Indopol® H-6000, Indopol® H-18000, Panalane® L-14E, or Panalane® H-300E. In certain embodiments, the polybutene pressure-sensitive adhesive is Indopol® H-1900. In other embodiments, the polybutene pressure-sensitive adhesive is Panalane® H-300E.

Acrylate copolymers of interest include copolymers of various monomers, such as “soft” monomers, “hard” monomers, or “functional” monomers. The acrylate copolymers can be composed of a copolymer including bipolymer (i.e., made with two monomers), a terpolymer (i.e., made with three monomers), or a tetrapolymer (i.e., made with four monomers), or copolymers having greater numbers of monomers. The acrylate copolymers may be crosslinked or non-crosslinked. The polymers can be cross-linked by known methods to provide the desired polymers. The monomers from of the acrylate copolymers may include at least two or more exemplary components selected from acrylic acids, alkyl acrylates, methacrylates, copolymerizable secondary monomers, and monomers with functional groups.

Monomers (“soft” and “hard” monomers) may be methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. Additional examples of acrylic adhesive monomers are described in Satas, “Acrylic Adhesives,” Handbook of Pressure-Sensitive Adhesive Technology, 2nd ed., pp. 396-456 (D. Satas, ed.), Van Nostrand Reinhold, New York (1989), the disclosure of which is herein incorporated by reference.

In certain embodiments, the pressure sensitive adhesive comprises a polyvinyl acetate copolymer. In certain embodiments, the pressure sensitive adhesive comprises ethylene-vinyl acetate copolymer, vinyl acetate-acrylic acid, polyvinyl chloride acetate, or polyvinylpyrrolidone. In certain embodiments, the pressure sensitive adhesive is an acrylate-vinyl acetate copolymer. In certain embodiments, the pressure sensitive adhesive may include a composition that is, or is substantially the same as, the composition of Duro-Tak® 87-9301, Duro-Tak® 87-200A, Duro-Tak®87-2353, Duro-Tak®87-2100, Duro-Tak®87-2051, Duro-Tak®87-2052, Duro-Tak®87-2194, Duro-Tak®87-2677, Duro-Tak®87-201A, Duro-Tak®87-2979, Duro-Tak®87-2510, Duro-Tak®87-2516, Duro-Tak®87-387, Duro-Tak®87-4287, Duro-Tak®87-2287, or Duro-Tak®87-2074. The term “substantially the same” as used in this context refers to a composition that is an acrylate-vinyl acetate copolymer in an organic solvent solution.

In certain embodiments, the pressure sensitive adhesive is an acrylate adhesive that is a non-functionalized acrylate, hydroxyl-functionalized acrylate, or an acid functionalized acrylate. For example, the acrylate adhesive may be an acrylic adhesive having one or more-OH functional groups. Where the acrylic adhesive has one or more-OH functional groups, in some instances, the pressure sensitive adhesive may be a composition that is, or is substantially the same as, the composition of Duro-Tak® 87-4287, Duro-Tak® 87-2287, Duro-Tak® 87-2510 or Duro-Tak® 87-2516. The acrylate adhesive may alternatively be an acrylic adhesive having one or more-COOH functional groups. Where the acrylic adhesive has one or more-COOH functional groups, in some instances, the pressure sensitive adhesive may be a composition that is or is substantially the same as, the composition of Duro-Tak® 87-387, Duro-Tak® 87-2979 or Duro-Tak® 87-2353. Still further, the acrylate adhesive may be a non-functionalized acrylic adhesive. Where the acrylic adhesive is non-functionalized, in some instances, the pressure sensitive adhesive may be a composition that is or is substantially the same as, the composition of Duro-Tak® 87-9301.

In certain embodiments, the pressure sensitive adhesive comprises an acrylic polymer, acrylate copolymer, acrylate-vinyl acetate copolymer, polyacrylonitrile, or a combination thereof.

In certain embodiments, the pressure sensitive adhesive comprises a hydroxyl functionalized acrylate copolymer.

The amount of pressure sensitive adhesive in the drug layer may vary, for example, the amount of pressure sensitive adhesive ranging from 0.1 mg to 2000 mg, such as, for example, from 0.5 mg to 1500 mg, from 1 to 1000 mg, from 10 to 750 mg, or from 10 mg to 500 mg. As such, in certain embodiments, the amount of pressure sensitive adhesive ranges from 1% to 99% (w/w), such as, for example, from 5% to 95% (w/w), from 10% to 95% (w/w), from 15% to 90% (w/w), or from 20% to 85% (w/w). In other embodiments, the amount of pressure sensitive adhesive in the drug layer is 70% by weight or greater, e.g., 75% by weight or greater, 80% by weight or greater, 85% by weight or greater, 90% by weight or greater, 95% by weight or greater, 97% by weight or greater.

The weight ratio of pressure sensitive adhesive to dexmedetomidine in the drug layer may range between 1:2 and 1:2.5; 1:2.5 and 1:3; 1:3 and 1:3.5; 1:3.5 and 1:4; 1:4 and 1:4.5; 1:4.5 and 1:5; 1:5 and 1:10; 1:10 and 1:25; 1:25 and 1:50; 1:50 and 1:75; and 1:75 and 1:99 or a range thereof. For example, the weight ratio of pressure sensitive adhesive to dexmedetomidine in the drug layer may range between 1:1 and 1:5; 1:5 and 1:10; 1:10 and 1:15; 1:15 and 1:25; 1:25 and 1:50; 1:50 and 1:75; or 1:75 and 1:99. Alternatively, the weight ratio of dexmedetomidine to pressure sensitive adhesive in the drug layer may range between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; 10:1 and 25:1; 25:1 and 50:1; 50:1 and 75:1; and 75:1 and 99:1 or a range thereof. For example, the ratio of dexmedetomidine to pressure sensitive adhesive in drug layer may range between 1:1 and 5:1; 5:1 and 10:1; 10:1 and 15:1; 15:1 and 25:1; 25:1 and 50:1; 50:1 and 75:1; or 75:1 and 99:1.

In certain embodiments, the drug layer may further include one or more crosslinked hydrophilic polymers. For example, the crosslinked polymer may be an amine-containing hydrophilic polymer. Amine-containing polymers include, but are not limited to, polyethyleneimine, amine-terminated polyethylene oxide, amine-terminated polyethylene/polypropylene oxide, polymers of dimethyl amino ethyl methacrylate, and copolymers of dimethyl amino ethyl methacrylate and vinyl pyrrolidone. In certain embodiments, the crosslinked polymer is crosslinked polyvinylpyrrolidone, such as, for example, PVP-CLM.

In certain embodiments, the drug layer may contain other additives depending on the adhesive used. For example, materials, such as PVP-CLM, PVP K17, PVP K30, PVP K90, that inhibit drug crystallization, have hygroscopic properties that improve the duration of wear, and improve the physical properties, e.g., cold flow, tack, cohesive strength, of the adhesive.

In certain embodiments, the amount of crosslinked polymer in the drug layer may vary. For example, the amount of crosslinked polymer may range from 0.1 mg to 500 mg, such as, for example, from 0.5 mg to 400 mg, from 1 to 300 mg, from 10 to 200 mg, or from 10 mg to 100 mg. As such, in certain embodiments, the amount of crosslinked polymer in the drug layer ranges from 2% to 30% (w/w), such as, for example, from 4% to 30% (w/w), from 5% to 25% (w/w), from 6% to 22.5% (w/w), or from 10% to 20% (w/w). In other embodiments, the amount of crosslinked polymer in the drug layer is 8% by weight or greater, such as, for example, 10% by weight or greater, 12% by weight or greater, 15% by weight or greater, 20% by weight or greater, 25% by weight or greater, or 30% by weight or greater.

In certain embodiments, the drug layer further comprises a permeation enhancer. Permeation enhancers increase the dexmedetomidine solubility, such as, for example, to prevent unwanted crystallization of dexmedetomidine in the drug layer. The permeation enhancer may be incorporated into the drug layer in an amount ranging from 0.01% to 20% (w/w), such as, for example, from 0.05% to 15% (w/w), from 0.1% to 10% (w/w), from 0.5% to 8% (w/w) or from 0.5% to 5% (w/w).

Exemplary permeation enhancers include, but are not limited to, acids including linolic acid, oleic acid, linolenic acid, stearic acid, isostearic acid, levulinic acid, palmitic acid, octanoic acid, decanoic acid, dodecanoic acid, tetradecanoic acid, hexadecanoic acid, octadecanoic acid, N-lauroyl sarcosine, L-pyroglutamic acid, lauric acid, succinic acid, pyruvic acid, glutaric acid, sebacic acid, cyclopentane carboxylic acid, and acylated amino acids. Other permeation enhancers of interest include, but are not limited to, aliphatic alcohols, such as saturated or unsaturated higher alcohols having 12 to 22 carbon atoms (e.g., oleyl alcohol or lauryl alcohol); fatty acid esters, such as isopropyl myristate, diisopropyl adipate, lauryl lactate, propyl laurate, ethyl oleate and isopropyl palmitate; alcohol amines, such as triethanolamine, triethanolamine hydrochloride, and diisopropanolamine; polyhydric alcohol alkyl ethers, such as alkyl ethers of polyhydric alcohols such as glycerol, ethylene glycol, propylene glycol, 1,3-butylene glycol, diglycerol, polyglycerol, diethylene glycol, polyethylene glycol, dipropylene glycol, polypropylene glycol, polypropylene glycolmonolaurate, sorbitan, sorbitol, isosorbide, methyl glucoside, oligosaccharides, and reducing oligosaccharides, where the number of carbon atoms of the alkyl group moiety in the polyhydric alcohol alkyl ethers is preferably 6 to 20; polyoxyethylene alkyl ethers, such as polyoxyethylene alkyl ethers in which the number of carbon atoms of the alkyl group moiety is 6 to 20, and the number of repeating units (e.g., —O-CH₂CH₂—) of the polyoxyethylene chain is 1 to 9, such as but not limited to polyoxyethylene lauryl ether, polyoxyethylene cetyl ether, polyoxyethylene stearyl ether, and polyoxyethylene oleyl ether; glycerides (i.e., fatty acid esters of glycerol), such as glycerol esters of fatty acids having 6 to 18 carbon atoms, where the glycerides may be monoglycerides (i.e., a glycerol molecule covalently bonded to one fatty acid chain through an ester linkage), diglycerides (i.e., a glycerol molecule covalently bonded to two fatty acid chains through ester linkages), triglycerides (i.e., a glycerol molecule covalently bonded to three fatty acid chains through ester linkages), or combinations thereof, where the fatty acid components forming the glycerides include octanoic acid, decanoic acid, dodecanoic acid, tetradecanoic acid, hexadecanoic acid, octadecanoic acid (i.e., stearic acid) and oleic acid; middle-chain fatty acid esters of polyhydric alcohols; lactic acid alkyl esters; dibasic acid alkyl esters; acylated amino acids; pyrrolidone; pyrrolidone derivatives and combinations thereof. Additional permeation enhancers may include lactic acid, tartaric acid, 1,2,6-hexanetriol, benzyl alcohol, lanoline, potassium hydroxide (KOH), tris(hydroxymethyl)aminomethane, glycerol monooleate (GMO), sorbitan monolaurate (SML), sorbitan monooleate (SMO), laureth-4 (LTH), and combinations thereof. In certain embodiments, the permeation enhancer comprises levulinic acid, lauryl lactate, oleic acid, propylene glycolmonolaurate, or a combination thereof.

In certain embodiments, the permeation enhancer comprises lauryl lactate, oleic acid, or a combination thereof. In certain embodiments, the permeation enhancer is lauryl lactate.

The drug layer of the transdermal delivery patch is affixed (e.g., by lamination) to a backing layer. In other words, the backing layer is in direct contact with the drug layer. The backing may be flexible so that it can be brought into close contact with the desired application site on the subject. In certain embodiments, the backing is fabricated from a material that does not absorb the dexmedetomidine and does not allow the dexmedetomidine to be leached from the matrix. Backing layers of interest may include, but are not limited to, non-woven fabrics, woven fabrics, films (including sheets), porous bodies, foamed bodies, paper, composite materials obtained by laminating a film on a non-woven fabric or fabric, and combinations thereof.

In certain embodiments, the backing layer comprises a film comprising a polyolefin resin, polyacrylic resin, polyester resin, cellophane, polyvinyl alcohol, ethylene-vinyl alcohol copolymer, polyvinyl chloride, polystyrene, polyurethane, polyacrylonitrile, fluororesin, styrene-isoprene-styrene copolymer, styrene-butadiene rubber, polybutadiene, ethylene-vinyl acetate copolymer, polyamide, polysulfone, or a combination thereof. In certain embodiments, the film comprises a polyolefin resin selected from polyethylene and polypropylene. In certain embodiments, the polyester resin is polyethylene terephthalate. In certain embodiments, the film comprises polyethylene and polyethylene terephthalate.

Non-woven fabrics may include polyolefin resins such as polyethylene and polypropylene; polyester resins such as polyethylene terephthalate, polybutylene terephthalate and polyethylene naphthalate; rayon, polyamide, poly(ester ether), polyurethane, polyacrylic resins, polyvinyl alcohol, styrene-isoprene-styrene copolymers, and styrene-ethylene-propylene-styrene copolymers; and combinations thereof.

Woven fabrics may include cotton, rayon, polyacrylic resins, polyester resins, polyvinyl alcohol, and combinations thereof.

Films may include polyolefin resins such as polyethylene and polypropylene; polyacrylic resins such as polymethyl methacrylate and polyethyl methacrylate; polyester resins such as polyethylene terephthalate, polybutylene terephthalate and polyethylene naphthalate; and besides cellophane, polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof. In certain embodiments, the film comprises polyethylene and polyethylene terephthalate.

Papers may include impregnated paper, coated paper, wood free paper, Kraft paper, Japanese paper, glassine paper, synthetic paper, and combinations thereof.

The surface of the drug layer opposite the backing layer is adhesive and adheres to a skin surface of a subject. In certain embodiments, the adhesive surface has a surface area from 3 cm² to 20 cm², such as, for example, from 3 cm² to 19 cm², from 3 cm² to 18 cm², from 3 cm² to 17 cm², from 3 cm² to 16 cm², from 3 cm² to 15 cm², from 3 cm² to 14 cm², from 3 cm² to 13 cm², from 3 cm² to 12 cm², from 3 cm² to 11 cm², from 3 cm² to 10 cm², from 3 cm² to 9 cm², from 3 cm² to 8 cm², from 3 cm² to 7 cm², from 3 cm² to 6 cm², from 3 cm² to 5 cm², from 3 cm² to 4 cm², from 4 cm² to 20 cm², from 4 cm² to 19 cm², from 4 cm² to 18 cm², from 4 cm² to 17 cm², from 4 cm² to 16 cm², from 4 cm² to 15 cm², from 4 cm² to 14 cm², from 4 cm² to 13 cm², from 4 cm² to 12 cm², from 4 cm² to 11 cm², from 4 cm² to 10 cm², from 4 cm² to 9 cm², from 4 cm² to 8 cm², from 4 cm² to 7 cm², from 4 cm² to 6 cm², from 4 cm² to 5 cm², from 5 cm² to 20 cm², from 5 cm² to 19 cm², from 5 cm² to 18 cm², from 5 cm² to 17 cm², from 5 cm² to 16 cm², from 5 cm² to 15 cm², from 5 cm² to 14 cm², from 5 cm² to 13 cm², from 5 cm² to 12 cm², from 5 cm² to 11 cm², from 5 cm² to 10 cm², from 5 cm² to 9 cm², from 5 cm² to 8 cm², from 5 cm² to 7 cm², from 5 cm² to 6 cm², from 6 cm² to 20 cm², from 6 cm² to 19 cm², from 6 cm² to 18 cm², from 6 cm² to 17 cm², from 6 cm² to 16 cm², from 6 cm² to 15 cm², from 6 cm² to 14 cm², from 6 cm² to 13 cm², from 6 cm² to 12 cm², from 6 cm² to 11 cm², from 6 cm² to 10 cm², from 6 cm² to 9 cm², from 6 cm² to 8 cm², from 6 cm² to 7 cm², from 7 cm² to 20 cm², from 7 cm² to 19 cm², from 7 cm² to 18 cm², from 7 cm² to 17 cm², from 7 cm² to 16 cm², from 7 cm² to 15 cm², from 7 cm² to 14 cm², from 7 cm² to 13 cm², from 7 cm² to 12 cm², from 7 cm² to 11 cm², from 7 cm² to 10 cm², from 7 cm² to 9 cm², from 7 cm² to 8 cm², from 8 cm² to 20 cm², from 8 cm² to 19 cm², from 8 cm² to 18 cm², from 8 cm² to 17 cm², from 8 cm² to 16 cm², from 8 cm² to 15 cm², from 8 cm² to 14 cm², from 8 cm² to 13 cm², from 8 cm² to 12 cm², from 8 cm² to 11 cm², from 8 cm² to 10 cm², from 8 cm² to 9 cm², from 9 cm² to 20 cm², from 9 cm² to 19 cm², from 9 cm² to 18 cm², from 9 cm² to 17 cm², from 9 cm² to 16 cm², from 9 cm² to 15 cm², from 9 cm² to 14 cm², from 9 cm² to 13 cm², from 9 cm² to 12 cm², from 9 cm² to 11 cm², from 9 cm² to 10 cm², from 10 cm² to 20 cm², from 10 cm² to 19 cm², from 10 cm² to 18 cm², from 10 cm² to 17 cm², from 10 cm² to 16 cm², from 10 cm² to 15 cm², from 10 cm² to 14 cm², from 10 cm² to 13 cm², from 10 cm² to 12 cm², from 10 cm² to 11 cm², from 11 cm² to 20 cm², from 11 cm² to 19 cm², from 11 cm² to 18 cm², from 11 cm² to 17 cm², from 11 cm² to 16 cm², from 11 cm² to 15 cm², from 11 cm² to 14 cm², from 11 cm² to 13 cm², from 11 cm² to 12 cm², from 12 cm² to 20 cm², from 12 cm² to 19 cm², from 12 cm² to 18 cm², from 12 cm² to 17 cm², from 12 cm² to 16 cm², from 12 cm² to 15 cm², from 12 cm² to 14 cm², from 12 cm² to 13 cm², from 13 cm² to 14 cm², from 14 cm² to 20 cm², from 14 cm² to 19 cm², from 14 cm² to 18 cm², from 14 cm² to 17 cm², from 14 cm² to 16 cm², from 14 cm² to 15 cm², from 15 cm² to 20 cm², from 15 cm² to 19 cm², from 15 cm² to 18 cm², from 15 cm² to 17 cm², 15 cm² to 16 cm², from 16 cm² to 20 cm², from 16 cm² to 19 cm², from 16 cm² to 18 cm², from 16 cm² to 17 cm², from 17 cm² to 20 cm², from 17 cm² to 19 cm², from 17 cm² to 18 cm², from 18 cm² to 20 cm², from 18 cm² to 19 cm² or from 19 cm² to 20 cm².

In certain embodiments, the surface area of the adhesive surface is from 3 cm² to 15 cm² and the drug layer comprises from 1 mg to 4 mg dexmedetomidine. In certain embodiments, the surface area of the adhesive surface is from 4 cm² to 15 cm² and the drug layer comprises from 1 mg to 4 mg dexmedetomidine. In certain embodiments, the surface area of the adhesive surface is from 5 cm² to 15 cm² and the drug layer comprises from 1 mg to 4 mg dexmedetomidine. In certain embodiments, the surface area of the adhesive surface is from 6 cm² to 15 cm² and the drug layer comprises from 1 mg to 4 mg dexmedetomidine. In certain embodiments, the surface area of the adhesive surface is from 7 cm² to 15 cm² and the drug layer comprises from 1 mg to 4 mg dexmedetomidine. In certain embodiments, the surface area of the adhesive surface is from 8 cm² to 15 cm² and the drug layer comprises from 1 mg to 4 mg dexmedetomidine.

In certain embodiments, the surface area of the adhesive surface is from 3 cm² to 8 cm² and the drug layer comprises from 1 mg to 3.5 mg dexmedetomidine. In certain embodiments, the surface area of the adhesive surface is from 4 cm² to 8 cm² and the drug layer comprises from 1 mg to 3.5 mg dexmedetomidine. In certain embodiments, the surface area of the adhesive surface is from 5 cm² to 8 cm² and the drug layer comprises from 1 mg to 3.5 mg dexmedetomidine. In certain embodiments, the surface area of the adhesive surface is from 6 cm² to 8 cm² and the drug layer comprises from 1 mg to 3.5 mg dexmedetomidine. In certain embodiments, the surface area of the adhesive surface is from 7 cm² to 8 cm² and the drug layer comprises from 1 mg to 3.5 mg dexmedetomidine. In certain embodiments, the surface area of the adhesive surface is from 3 cm² to 7 cm² and the drug layer comprises from 1 mg to 3.5 mg dexmedetomidine. In certain embodiments, the surface area of the adhesive surface is from 4 cm² to 7 cm² and the drug layer comprises from 1 mg to 3.5 mg dexmedetomidine. In certain embodiments, the surface area of the adhesive surface is from 5 cm² to 7 cm² and the drug layer comprises from 1 mg to 3.5 mg dexmedetomidine. In certain embodiments, the surface area of the adhesive surface is from 6 cm² to 7 cm² and the drug layer comprises from 1 mg to 3.5 mg dexmedetomidine. In certain embodiments, the surface area of the adhesive surface is from 3 cm² to 6 cm² and the drug layer comprises from 1 mg to 3.5 mg dexmedetomidine. In certain embodiments, the surface area of the adhesive surface is from 4 cm² to 6 cm² and the drug layer comprises from 1 mg to 3.5 mg dexmedetomidine. In certain embodiments, the surface area of the adhesive surface is from 5 cm² to 6 cm² and the drug layer comprises from 1 mg to 3.5 mg dexmedetomidine. In certain embodiments, the surface area of the adhesive surface is from 3 cm² to 5 cm² and the drug layer comprises from 1 mg to 3.5 mg dexmedetomidine. In certain embodiments, the surface area of the adhesive surface is from 4 cm² to 5 cm² and the drug layer comprises from 1 mg to 3.5 mg dexmedetomidine. In certain embodiments, the surface area of the adhesive surface is from 3 cm² to 4 cm² and the drug layer comprises from 1 mg to 3.5 mg dexmedetomidine. In certain embodiments, the surface area of the adhesive surface is 6 cm² and the drug layer comprises from 1 mg to 3.5 mg dex medetomidine.

In certain embodiments, the surface area of the adhesive surface is from 3 cm² to 15 cm² and the drug layer comprises from 1 mg to 4 mg dexmedetomidine. In certain embodiments, the surface area of the adhesive surface is from 3 cm² to 15 cm² and the drug layer comprises from 1 mg to 2 mg dexmedetomidine. In certain embodiments, the surface area of the adhesive surface is from 3 cm² to 15 cm² and the drug layer comprises from 1.3 mg to 1.6 mg dexmedetomidine.

In certain embodiments, the surface area of the adhesive surface is from 6 cm² to 7 cm² and the drug layer comprises 1.4 to 1.5 mg dexmedetomidine, e.g., 1.46 mg dexmedetomidine. In certain embodiments, the surface area of the adhesive surface is from 9 cm² to 10 cm² and the drug layer comprises 1.4 to 1.5 mg dexmedetomidine, e.g., 1.46 mg dexmedetomidine. In certain embodiments, the surface area of the adhesive surface is from 12 cm² to 13 cm² and the drug layer comprises 1.4 to 1.5 mg dexmedetomidine, e.g., 1.46 mg dexmedetomidine.

In certain embodiments, the surface area of the adhesive surface is from 6 cm² to 7 cm² and the drug layer comprises 2 to 3 mg dexmedetomidine, e.g., 2.92 mg dexmedetomidine. In certain embodiments, the surface area of the adhesive surface is from 9 cm² to 10 cm² and the drug layer comprises 2 to 3 mg dexmedetomidine, e.g., 2.92 mg dexmedetomidine. In certain embodiments, the surface area of the adhesive surface is from 12 cm² to 13 cm² and the drug layer comprises 2 to 3 mg dexmedetomidine, e.g., 2.92 mg dexmedetomidine. In certain embodiments, the surface area of the adhesive surface is from 15 cm² to 16 cm² and the drug layer comprises 3 to 4 mg dexmedetomidine, e.g., 3.65 mg dexmedetomidine.

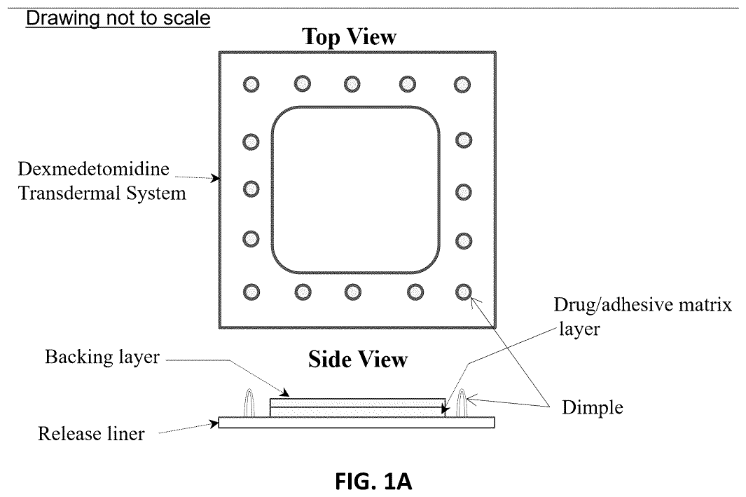

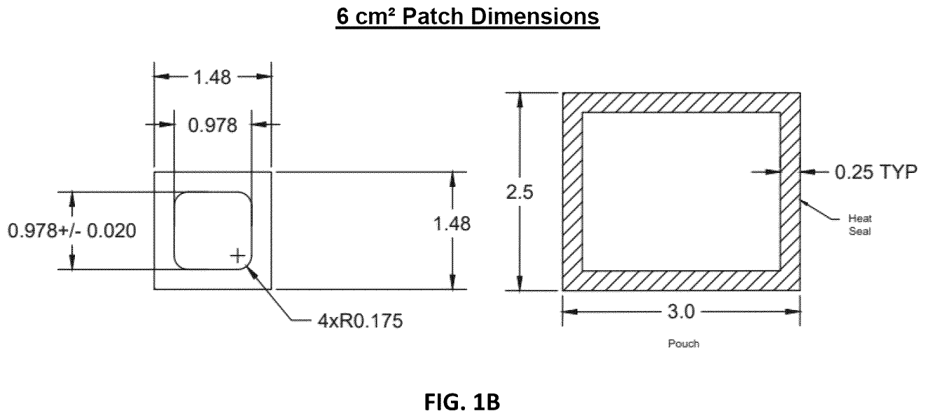

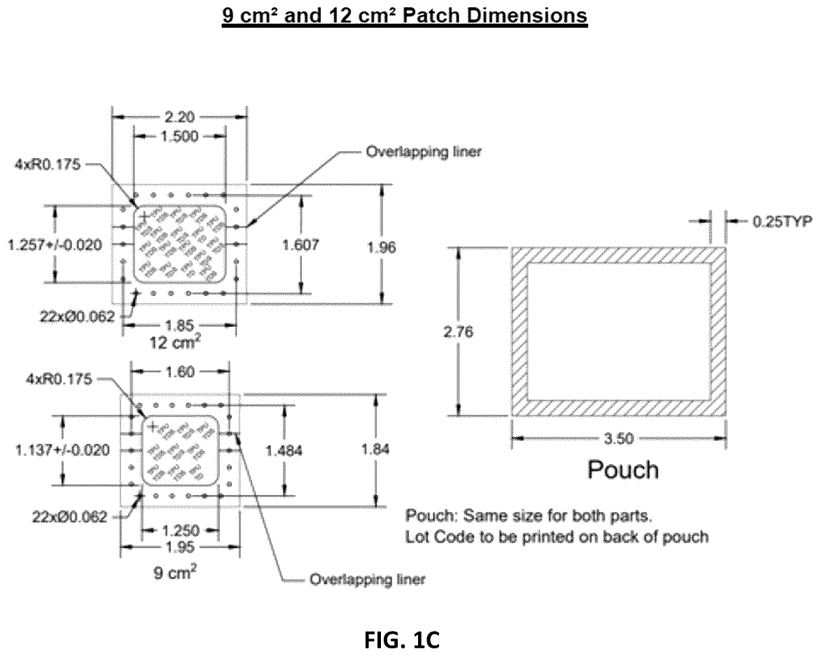

The transdermal delivery patch can be any shape and size so long as the surface area of the adhesive surface is as provided herein. In certain embodiments, the transdermal delivery patch is rectangular. In certain embodiments, the transdermal delivery patch is rectangular with rounded corners. In certain embodiments, the transdermal delivery patch is square. In certain embodiments, the transdermal delivery patch is square with rounded corners. In certain embodiments, the transdermal delivery patch is circular. In certain embodiments, the transdermal delivery patch is oval. Exemplary transdermal delivery patches are depicted in FIGS. 1A-C.

In certain embodiments, the transdermal delivery patch has a length of 0.5 inches to 4 inches and a width of 0.5 inches to 4 inches, such as, for example, a length from 1 inch to 4 inches and a width from 0.5 inches to 4 inches, a length from 1.5 inches to 4 inches and a width from 0.5 inches to 4 inches, a length from 2 inches to 4 inches and a width from 0.5 inches to 4 inches, a length from 2.5 inches to 4 inches and a width from 0.5 inches to 4 inches, a length from 3 inches to 4 inches and a width from 0.5 inches to 4 inches, a length from 3.5 inches to 4 inches and a width from 0.5 inches to 4 inches, a length of 0.5 inches to 4 inches and a width of 0.5 inches to 3.5 inches, a length from 1 inch to 4 inches and a width from 0.5 inches to 3.5 inches, a length from 1.5 inches to 4 inches and a width from 0.5 inches to 3.5 inches, a length from 2 inches to 4 inches and a width from 0.5 inches to 3.5 inches, a length from 2.5 inches to 4 inches and a width from 0.5 inches to 3.5 inches, a length from 3 inches to 4 inches and a width from 0.5 inches to 3.5 inches, a length from 3.5 inches to 4 inches and a width from 0.5 inches to 3.5 inches, a length of 0.5 inches to 4 inches and a width of 0.5 inches to 3 inches, a length from 1 inch to 4 inches and a width from 0.5 inches to 3 inches, a length from 1.5 inches to 4 inches and a width from 0.5 inches to 3 inches, a length from 2 inches to 4 inches and a width from 0.5 inches to 3 inches, a length from 2.5 inches to 4 inches and a width from 0.5 inches to 3 inches, a length from 3 inches to 4 inches and a width from 0.5 inches to 3 inches, a length from 3.5 inches to 4 inches and a width from 0.5 inches to 3 inches, a length of 0.5 inches to 4 inches and a width of 0.5 inches to 2.5 inches, a length from 1 inch to 4 inches and a width from 0.5 inches to 2.5 inches, a length from 1.5 inches to 4 inches and a width from 0.5 inches to 2.5 inches, a length from 2 inches to 4 inches and a width from 0.5 inches to 2.5 inches, a length from 2.5 inches to 4 inches and a width from 0.5 inches to 2.5 inches, a length from 3 inches to 4 inches and a width from 0.5 inches to 2.5 inches, a length from 3.5 inches to 4 inches and a width from 0.5 inches to 2.5 inches, a length of 0.5 inches to 4 inches and a width of 0.5 inches to 2 inches, a length from 1 inch to 4 inches and a width from 0.5 inches to 2 inches, a length from 1.5 inches to 4 inches and a width from 0.5 inches to 2 inches, a length from 2 inches to 4 inches and a width from 0.5 inches to 2 inches, a length from 2.5 inches to 4 inches and a width from 0.5 inches to 2 inches, a length from 3 inches to 4 inches and a width from 0.5 inches to 2 inches, a length from 3.5 inches to 4 inches and a width from 0.5 inches to 2 inches, a length of 0.5 inches to 4 inches and a width of 0.5 inches to 1.5 inches, a length from 1 inch to 4 inches and a width from 0.5 inches to 1.5 inches, a length from 1.5 inches to 4 inches and a width from 0.5 inches to 1.5 inches, a length from 2 inches to 4 inches and a width from 0.5 inches to 1.5 inches, a length from 2.5 inches to 4 inches and a width from 0.5 inches to 1.5 inches, a length from 3 inches to 4 inches and a width from 0.5 inches to 1.5 inches, a length from 3.5 inches to 4 inches and a width from 0.5 inches to 1.5 inches, a length of 0.5 inches to 4 inches and a width of 0.5 inches to 1 inch, a length from 1 inch to 4 inches and a width from 0.5 inches to 1 inch, a length from 1.5 inches to 4 inches and a width from 0.5 inches to 1 inch, a length from 2 inches to 4 inches and a width from 0.5 inches to 1 inch, a length from 2.5 inches to 4 inches and a width from 0.5 inches to 1 inch, a length from 3 inches to 4 inches and a width from 0.5 inches to 1 inch, a length from 3.5 inches to 4 inches and a width from 0.5 inches to 1 inch, a length from 0.5 inches to 4 inches and a width from 1 inch to 4 inches, a length from 0.5 inches to 4 inches and a width from 1.5 inches to 4 inches, a length from 0.5 inches to 4 inches and a width from 2 inches to 4 inches, a length from 0.5 inches to 4 inches and a width from 2.5 inches to 4 inches, a length from 0.5 inches to 4 inches and a width from 3 inches to 4 inches, a length from 0.5 inches to 4 inches and a width from 3.5 inches to 4 inches, a length from 0.5 inches to 3.5 inches and a width from 0.5 inches to 4 inches, a length from 0.5 inches to 3.5 inches and a width from 1 inch to 4 inches, a length from 0.5 inches to 3.5 inches and a width from 1.5 inches to 4 inches, a length from 0.5 inches to 3.5 inches and a width from 2 inches to 4 inches, a length from 0.5 inches to 3.5 inches and a width from 2.5 inches to 4 inches, a length from 0.5 inches to 3.5 inches and a width from 3 inches to 4 inches, a length from 0.5 inches to 3.5 inches and a width from 3.5 inches to 4 inches, a length from 0.5 inches to 3 inches and a width from 0.5 inches to 4 inches, a length from 0.5 inches to 3 inches and a width from 1 inch to 4 inches, a length from 0.5 inches to 3 inches and a width from 1.5 inches to 4 inches, a length from 0.5 inches to 3 inches and a width from 2 inches to 4 inches, a length from 0.5 inches to 3 inches and a width from 2.5 inches to 4 inches, a length from 0.5 inches to 3 inches and a width from 3 inches to 4 inches, a length from 0.5 inches to 3 inches and a width from 3.5 inches to 4 inches, a length from 0.5 inches to 2.5 inches and a width from 0.5 inches to 4 inches, a length from 0.5 inches to 2.5 inches and a width from 1 inch to 4 inches, a length from 0.5 inches to 2.5 inches and a width from 1.5 inches to 4 inches, a length from 0.5 inches to 2.5 inches and a width from 2 inches to 4 inches, a length from 0.5 inches to 2.5 inches and a width from 2.5 inches to 4 inches, a length from 0.5 inches to 2.5 inches and a width from 3 inches to 4 inches, a length from 0.5 inches to 2.5 inches and a width from 3.5 inches to 4 inches, a length from 0.5 inches to 2 inches and a width from 0.5 inches to 4 inches, a length from 0.5 inches to 2 inches and a width from 1 inch to 4 inches, a length from 0.5 inches to 2 inches and a width from 1.5 inches to 4 inches, a length from 0.5 inches to 2 inches and a width from 2 inches to 4 inches, a length from 0.5 inches to 2 inches and a width from 2.5 inches to 4 inches, a length from 0.5 inches to 2 inches and a width from 3 inches to 4 inches, a length from 0.5 inches to 2 inches and a width from 3.5 inches to 4 inches, a length from 0.5 inches to 1.5 inches and a width from 0.5 inches to 4 inches, a length from 0.5 inches to 1.5 inches and a width from 1 inch to 4 inches, a length from 0.5 inches to 1.5 inches and a width from 1.5 inches to 4 inches, a length from 0.5 inches to 1.5 inches and a width from 2 inches to 4 inches, a length from 0.5 inches to 1.5 inches and a width from 2.5 inches to 4 inches, a length from 0.5 inches to 1.5 inches and a width from 3 inches to 4 inches, a length from 0.5 inches to 1.5 inches and a width from 3.5 inches to 4 inches, a length from 0.5 inches to 1 inch and a width from 0.5 inches to 4 inches, a length from 0.5 inches to 1 inch and a width from 1 inch to 4 inches, a length from 0.5 inches to 1 inch and a width from 1.5 inches to 4 inches, a length from 0.5 inches to 1 inch and a width from 2 inches to 4 inches, a length from 0.5 inches to 1 inch and a width from 2.5 inches to 4 inches, a length from 0.5 inches to 1 inch and a width from 3 inches to 4 inches, a length from 0.5 inches to 1 inch and a width from 3.5 inches to 4 inches.

In certain embodiments, the transdermal delivery patch has a length of 0.5 cm to 1 cm and a width of 0.5 cm to 1 cm.

In certain embodiments, the transdermal delivery patch has a length of 1 to 1.5 inches and a width of 1 inch to 1.5 inches.

In certain embodiments, the transdermal delivery patch has a length of 1.5 to 2 inches and a width of 1 to 1.5 inches.

In certain embodiments, the adhesive surface of the transdermal delivery patch is adhered to a release liner. In such embodiments, the drug layer is essentially sandwiched between the backing layer and the release liner. The release liner can be removed manually, thereby exposing the adhesive face of the drug layer of the transdermal delivery patch, which can then be adhered to a skin surface.

In certain embodiments, the adhesive surface of the transdermal delivery patch has a length of 0.5 inches to 4 inches and a width of 0.5 inches to 4 inches, such as, for example, a length from 1 inch to 4 inches and a width from 0.5 inches to 4 inches, a length from 1.5 inches to 4 inches and a width from 0.5 inches to 4 inches, a length from 2 inches to 4 inches and a width from 0.5 inches to 4 inches, a length from 2.5 inches to 4 inches and a width from 0.5 inches to 4 inches, a length from 3 inches to 4 inches and a width from 0.5 inches to 4 inches, a length from 3.5 inches to 4 inches and a width from 0.5 inches to 4 inches, a length of 0.5 inches to 4 inches and a width of 0.5 inches to 3.5 inches, a length from 1 inch to 4 inches and a width from 0.5 inches to 3.5 inches, a length from 1.5 inches to 4 inches and a width from 0.5 inches to 3.5 inches, a length from 2 inches to 4 inches and a width from 0.5 inches to 3.5 inches, a length from 2.5 inches to 4 inches and a width from 0.5 inches to 3.5 inches, a length from 3 inches to 4 inches and a width from 0.5 inches to 3.5 inches, a length from 3.5 inches to 4 inches and a width from 0.5 inches to 3.5 inches, a length of 0.5 inches to 4 inches and a width of 0.5 inches to 3 inches, a length from 1 inch to 4 inches and a width from 0.5 inches to 3 inches, a length from 1.5 inches to 4 inches and a width from 0.5 inches to 3 inches, a length from 2 inches to 4 inches and a width from 0.5 inches to 3 inches, a length from 2.5 inches to 4 inches and a width from 0.5 inches to 3 inches, a length from 3 inches to 4 inches and a width from 0.5 inches to 3 inches, a length from 3.5 inches to 4 inches and a width from 0.5 inches to 3 inches, a length of 0.5 inches to 4 inches and a width of 0.5 inches to 2.5 inches, a length from 1 inch to 4 inches and a width from 0.5 inches to 2.5 inches, a length from 1.5 inches to 4 inches and a width from 0.5 inches to 2.5 inches, a length from 2 inches to 4 inches and a width from 0.5 inches to 2.5 inches, a length from 2.5 inches to 4 inches and a width from 0.5 inches to 2.5 inches, a length from 3 inches to 4 inches and a width from 0.5 inches to 2.5 inches, a length from 3.5 inches to 4 inches and a width from 0.5 inches to 2.5 inches, a length of 0.5 inches to 4 inches and a width of 0.5 inches to 2 inches, a length from 1 inch to 4 inches and a width from 0.5 inches to 2 inches, a length from 1.5 inches to 4 inches and a width from 0.5 inches to 2 inches, a length from 2 inches to 4 inches and a width from 0.5 inches to 2 inches, a length from 2.5 inches to 4 inches and a width from 0.5 inches to 2 inches, a length from 3 inches to 4 inches and a width from 0.5 inches to 2 inches, a length from 3.5 inches to 4 inches and a width from 0.5 inches to 2 inches, a length of 0.5 inches to 4 inches and a width of 0.5 inches to 1.5 inches, a length from 1 inch to 4 inches and a width from 0.5 inches to 1.5 inches, a length from 1.5 inches to 4 inches and a width from 0.5 inches to 1.5 inches, a length from 2 inches to 4 inches and a width from 0.5 inches to 1.5 inches, a length from 2.5 inches to 4 inches and a width from 0.5 inches to 1.5 inches, a length from 3 inches to 4 inches and a width from 0.5 inches to 1.5 inches, a length from 3.5 inches to 4 inches and a width from 0.5 inches to 1.5 inches, a length of 0.5 inches to 4 inches and a width of 0.5 inches to 1 inch, a length from 1 inch to 4 inches and a width from 0.5 inches to 1 inch, a length from 1.5 inches to 4 inches and a width from 0.5 inches to 1 inch, a length from 2 inches to 4 inches and a width from 0.5 inches to 1 inch, a length from 2.5 inches to 4 inches and a width from 0.5 inches to 1 inch, a length from 3 inches to 4 inches and a width from 0.5 inches to 1 inch, a length from 3.5 inches to 4 inches and a width from 0.5 inches to 1 inch, a length from 0.5 inches to 4 inches and a width from 1 inch to 4 inches, a length from 0.5 inches to 4 inches and a width from 1.5 inches to 4 inches, a length from 0.5 inches to 4 inches and a width from 2 inches to 4 inches, a length from 0.5 inches to 4 inches and a width from 2.5 inches to 4 inches, a length from 0.5 inches to 4 inches and a width from 3 inches to 4 inches, a length from 0.5 inches to 4 inches and a width from 3.5 inches to 4 inches, a length from 0.5 inches to 3.5 inches and a width from 0.5 inches to 4 inches, a length from 0.5 inches to 3.5 inches and a width from 1 inch to 4 inches, a length from 0.5 inches to 3.5 inches and a width from 1.5 inches to 4 inches, a length from 0.5 inches to 3.5 inches and a width from 2 inches to 4 inches, a length from 0.5 inches to 3.5 inches and a width from 2.5 inches to 4 inches, a length from 0.5 inches to 3.5 inches and a width from 3 inches to 4 inches, a length from 0.5 inches to 3.5 inches and a width from 3.5 inches to 4 inches, a length from 0.5 inches to 3 inches and a width from 0.5 inches to 4 inches, a length from 0.5 inches to 3 inches and a width from 1 inch to 4 inches, a length from 0.5 inches to 3 inches and a width from 1.5 inches to 4 inches, a length from 0.5 inches to 3 inches and a width from 2 inches to 4 inches, a length from 0.5 inches to 3 inches and a width from 2.5 inches to 4 inches, a length from 0.5 inches to 3 inches and a width from 3 inches to 4 inches, a length from 0.5 inches to 3 inches and a width from 3.5 inches to 4 inches, a length from 0.5 inches to 2.5 inches and a width from 0.5 inches to 4 inches, a length from 0.5 inches to 2.5 inches and a width from 1 inch to 4 inches, a length from 0.5 inches to 2.5 inches and a width from 1.5 inches to 4 inches, a length from 0.5 inches to 2.5 inches and a width from 2 inches to 4 inches, a length from 0.5 inches to 2.5 inches and a width from 2.5 inches to 4 inches, a length from 0.5 inches to 2.5 inches and a width from 3 inches to 4 inches, a length from 0.5 inches to 2.5 inches and a width from 3.5 inches to 4 inches, a length from 0.5 inches to 2 inches and a width from 0.5 inches to 4 inches, a length from 0.5 inches to 2 inches and a width from 1 inch to 4 inches, a length from 0.5 inches to 2 inches and a width from 1.5 inches to 4 inches, a length from 0.5 inches to 2 inches and a width from 2 inches to 4 inches, a length from 0.5 inches to 2 inches and a width from 2.5 inches to 4 inches, a length from 0.5 inches to 2 inches and a width from 3 inches to 4 inches, a length from 0.5 inches to 2 inches and a width from 3.5 inches to 4 inches, a length from 0.5 inches to 1.5 inches and a width from 0.5 inches to 4 inches, a length from 0.5 inches to 1.5 inches and a width from 1 inch to 4 inches, a length from 0.5 inches to 1.5 inches and a width from 1.5 inches to 4 inches, a length from 0.5 inches to 1.5 inches and a width from 2 inches to 4 inches, a length from 0.5 inches to 1.5 inches and a width from 2.5 inches to 4 inches, a length from 0.5 inches to 1.5 inches and a width from 3 inches to 4 inches, a length from 0.5 inches to 1.5 inches and a width from 3.5 inches to 4 inches, a length from 0.5 inches to 1 inch and a width from 0.5 inches to 4 inches, a length from 0.5 inches to 1 inch and a width from 1 inch to 4 inches, a length from 0.5 inches to 1 inch and a width from 1.5 inches to 4 inches, a length from 0.5 inches to 1 inch and a width from 2 inches to 4 inches, a length from 0.5 inches to 1 inch and a width from 2.5 inches to 4 inches, a length from 0.5 inches to 1 inch and a width from 3 inches to 4 inches, a length from 0.5 inches to 1 inch and a width from 3.5 inches to 4 inches.

In certain embodiments, the adhesive surface of the transdermal delivery patch has a length of 0.5 cm to 1 cm and a width of 0.5 cm to 1 cm.

In certain embodiments, the adhesive surface of the transdermal delivery patch has a length of 1 to 1.5 inches and a width of 1 inch to 1.5 inches.

In certain embodiments, the adhesive surface of the transdermal delivery patch has a length of 1.5 to 2 inches and a width of 1 to 1.5 inches.

In certain embodiments, the release liner is a silicon-coated or fluoropolymer-coated release liner. In certain embodiments, the release liner comprises a polyester sheet. In certain embodiments, the release liner comprises a silicon-coated polyester sheet. In certain embodiments, the release liner comprises a fluoropolymer-coated polyester sheet.

In certain embodiments, the one or more transdermal delivery patches are packaged within a pouch. Any suitable pouch material can be used, e.g., a single layer or multi-layer pouch. Exemplary pouch materials include, but are not limited to, polyesters, polyethylene, foil, and combinations thereof. In certain embodiments, the pouch comprises a polyester, e.g., polyethylene terephthalate. In certain embodiments, the pouch comprises polyethylene. In certain embodiments, the pouch comprises foil. In certain embodiments, the one or more transdermal delivery patches is packaged within a multi-layer pouch.

In embodiments, the pouch has a length of 1 inch to 4 inches and a width of 1 inch to 4 inches, such as, for example, a length 1.5 inches to 4 inches and a width of 1 inch to 4 inches, a length 2 inches to 4 inches and a width of 1 inch to 4 inches, a length 2.5 inches to 4 inches and a width of 1 inch to 4 inches, a length 3 inches to 4 inches and a width of 1 inch to 4 inches, a length 3.5 inches to 4 inches and a width of 1 inch to 4 inches, a length 1 inch to 3.5 inches and a width of 1 inch to 4 inches, a length 1.5 inches to 3.5 inches and a width of 1 inch to 4 inches, a length 2 inches to 3.5 inches and a width of 1 inch to 4 inches, a length 2.5 inches to 3.5 inches and a width of 1 inch to 4 inches, a length 3 inches to 3.5 inches and a width of 1 inch to 4 inches, a length 1 inch to 3 inches and a width of 1 inch to 4 inches, a length 1.5 inches to 3 inches and a width of 1 inch to 4 inches, a length 2 inches to 3 inches and a width of 1 inch to 4 inches, a length 2.5 inches to 3 inches and a width of 1 inch to 4 inches, a length 1 inch to 2.5 inches and a width of 1 inch to 4 inches, a length 1.5 inches to 2.5 inches and a width of 1 inch to 4 inches, a length 2 inches to 2.5 inches and a width of 1 inch to 4 inches, a length 1 inch to 2 inches and a width of 1 inch to 4 inches, a length 1.5 inches to 2 inches and a width of 1 inch to 4 inches, a length 1 inch to 1.5 inches and a width of 1 inch to 4 inches, a length of 1 inch to 4 inches and a width of 1 inch to 3.5 inches, a length of 1.5 inches to 4 inches and a width of 1 inch to 3.5 inches, a length of 1 inch to 4 inches and a width of 1 inch to 3.5 inches, a length of 1 inch to 4 inches and a width of 1.5 inches to 3.5 inches, a length of 1 inch to 4 inches and a width of 2 inches to 3.5 inches, a length of 1 inch to 4 inches and a width of 2.5 inches to 3.5 inches, a length of 1 inch to 4 inches and a width of 3 inches to 3.5 inches, a length of 1 inch to 4 inches and a width of 1 inch to 3 inches, a length of 1 inch to 4 inches and a width of 1.5 inches to 3 inches, a length of 1 inch to 4 inches and a width of 2 inches to 3 inches, a length of 1 inch to 4 inches and a width of 2.5 inches to 3 inches, a length of 1 inch to 4 inches and a width of 1 inch to 2.5 inches, a length of 1 inch to 4 inches and a width of 1.5 inches to 2.5 inches, a length of 1 inch to 4 inches and a width of 2 inches to 2.5 inches, a length of 1 inch to 4 inches and a width of 1 inch to 2 inches, a length of 1 inch to 4 inches and a width of 1.5 inches to 2 inches, a length of 1 inch to 4 inches and a width of 1 inch to 1.5 inches.

In certain embodiments, the pouch has a length of 2.75 to 3.5 inches and a width of 2 to 2.75 inches. In certain embodiments, the pouch has a length of 3.25 to 4 inches and a width of 2.5 to 3 inches.

In certain embodiments, the transdermal delivery patch comprises a drug layer affixed to a backing layer, wherein:
the drug layer comprises from 1 mg to 2 mg dexmedetomidine (e.g., from 1.3 mg to 1.6 mg dexmedetomidine or from 1.4 mg to 1.5 mg dexmedetomidine), a pressure sensitive adhesive comprising a hydroxyl functionalized acrylate polymer, and lauryl lactate;
the drug layer has an adhesive surface suitable for adhesion to a skin surface, wherein the adhesive face has a surface area from 3 cm² to 9 cm², such as from 6 cm² to 7 cm²; and
the backing layer comprises polyethylene and polyethylene terephthalate.

In certain embodiments, the transdermal delivery patch comprises a drug layer affixed to a backing layer, wherein:
the drug layer comprises from 1 mg to 2 mg dexmedetomidine (e.g., from 1.3 mg to 1.6 mg dexmedetomidine or from 1.4 mg to 1.5 mg dexmedetomidine), a pressure sensitive adhesive comprising a hydroxyl functionalized acrylate polymer, and lauryl lactate;
the drug layer is adhered to a silicon- or fluoropolymer-coated release liner, wherein the release liner, when removed, exposes an adhesive surface of the drug layer suitable for adhesion to a skin surface, wherein the adhesive face has a surface area from 3 cm² to 9 cm², such as from 6 cm² to 7 cm²; and
the backing layer comprises polyethylene and polyethylene terephthalate.

In certain embodiments, the transdermal delivery patch comprises a drug layer affixed to a backing layer, wherein:
the drug layer comprises from 2.5 mg to 3.5 mg dexmedetomidine (e.g., from 2.6 mg to 3.2 mg dexmedetomidine), a pressure sensitive adhesive comprising a hydroxyl functionalized acrylate polymer, and lauryl lactate;
the drug layer has an adhesive surface suitable for adhesion to a skin surface, wherein the adhesive face has a surface area from 7 cm² to 11 cm², such as from 9 cm² to 10 cm²; and
the backing layer comprises polyethylene and polyethylene terephthalate.

In certain embodiments, the transdermal delivery patch comprises a drug layer affixed to a backing layer, wherein:
the drug layer comprises from 2.5 mg to 3.5 mg dexmedetomidine (e.g., from 2.6 mg to 3.2 mg dexmedetomidine), a pressure sensitive adhesive comprising a hydroxyl functionalized acrylate polymer, and lauryl lactate;
the drug layer is adhered to a silicon- or fluoropolymer-coated release liner, wherein the release liner, when removed, exposes an adhesive surface of the drug layer suitable for adhesion to a skin surface, wherein the adhesive face has a surface area from 7 cm² to 11 cm², such as from 9 cm² to 10 cm²; and
the backing layer comprises polyethylene and polyethylene terephthalate.

In certain embodiments, the transdermal delivery patch comprises a drug layer affixed to a backing layer, wherein:
the drug layer comprises from 2.5 mg to 3.5 mg dexmedetomidine (e.g., from 2.6 mg to 3.2 mg dexmedetomidine), a pressure sensitive adhesive comprising a hydroxyl functionalized acrylate polymer, and lauryl lactate;
the drug layer has an adhesive surface suitable for adhesion to a skin surface, wherein the adhesive face has a surface area from 11 cm² to 14 cm², such as from 12 cm² to 13 cm²; and
the backing layer comprises polyethylene and polyethylene terephthalate.

In certain embodiments, the transdermal delivery patch comprises a drug layer affixed to a backing layer, wherein:
the drug layer comprises from 2.5 mg to 3.5 mg dexmedetomidine (e.g., from 2.6 mg to 3.2 mg dexmedetomidine), a pressure sensitive adhesive comprising a hydroxyl functionalized acrylate polymer, and lauryl lactate;
the drug layer is adhered to a silicon- or fluoropolymer-coated release liner, wherein the release liner, when removed, exposes an adhesive surface of the drug layer suitable for adhesion to a skin surface, wherein the adhesive face has a surface area from 11 cm² to 14 cm², such as from 12 cm² to 13 cm²; and
the backing layer comprises polyethylene and polyethylene terephthalate.

In certain embodiments, one transdermal delivery patch described herein is applied to the skin surface of the subject. In other embodiments, one or more transdermal delivery patches are applied to the skin surface of the subject, e.g., two, three, four, or five or more transdermal delivery patches.

In embodiments utilizing multiple transdermal delivery patches, the total dose of dexmedetomidine (e.g., 1 mg to 3.5 mg) can be divided equally or unequally between the transdermal delivery patches. In certain embodiments, the total dose of dexmedetomidine is divided equally. For example, a total dose of 2.92 mg dexmedetomidine can be achieved with two transdermal delivery patches described herein, wherein each patch comprises from 1.3 mg to 1.6 mg dexmedetomidine (e.g., 1.46 dexmedetomidine).

In certain embodiments when at least two transdermal delivery patches are used, they can be applied adjacent to one another on the skin surface of the subject. “Adjacent,” as used herein, means that the transdermal delivery patches are next to one another or adjoining one another.

### 5. EXAMPLES

The following abbreviations used in the examples have the definitions set forth below:

| Abbreviation or | |
|---|---|
| Specialized Term | Explanation |
| ABS | Agitated Behavior Scale |
| AD | Alzheimer's Disease/dementia of the Alzheimer's |
| | type |
| AE | adverse event |
| ANCOVA | analysis of covariance |
| BMI | body mass index |
| BP | blood pressure |
| CMAI | Cohen-Mansfield Agitation Inventory |
| CRF | case report form |
| C-SSRS | Columbia-Suicide Severity Rating Scale |
| DMTS | dexmedetomidine transdermal system |
| EAS-NH | Episodic Agitation Scale-Nursing Home |
| ECG | electrocardiogram |
| EOS | End of Study |
| ET | Early Termination |
| FDA | Food and Drug Administration |
| FSH | follicle stimulating hormone |
| GCP | Good Clinical Practice |
| HbsAg | hepatitis B surface antigen |
| HCV Ab | hepatitis C virus antibody |
| HEENT | head, ears, eyes, nose, and throat |
| HIV | human immunodeficiency virus |
| HR | heart rate |
| ICF | Informed Consent Form |
| IR | immediate release |
| IV | intravenous |
| IWRS | interactive web response system |
| LAR | Legally authorized representative |
| LOCF | last observation carried forward |
| MedDRA | Medical Dictionary for Regulatory Activities |
| MMSE | Mini-Mental State Examination |
| MTD | maximum tolerated dose |
| NIA-AA | National Institute on Aging and the Alzheimer's |
| | Association |
| NPI-NH | Neuropsychiatric Inventory - Nursing Home |
| | Version |
| NRSSPI | numeric rating scale of summed pain intensity |
| PE | physical examination |
| PK | pharmacokinetic(s) |
| RR | respiratory rate |
| SAE | serious adverse event |
| SAP | Statistical Analysis Plan |
| ABS | Agitated Behavior Scale |
| SD | standard deviation |
| SMC | Safety Monitoring Committee |
| SOC | system organ class |
| SPI | summed pain intensity |
| SpO₂ | oxygen saturation |
| WBC | white blood cell |

### Example 1: Preparation of an Exemplary Transdermal Delivery Patch

Formulations were prepared by mixing dexmedetomidine and a pressure sensitive adhesive in organic solvents (e.g., 30-60 wt % solid content in ethyl acetate, isopropyl alcohol, hexane, or heptane), followed by mixing. Once a homogeneous mixture was formed, the solution was cast on a release liner (siliconized polyester or fluoropolymer coated polyester sheets of 2-3 mils) and dried at 60°−80° C. for 10-90 minutes. The single layer adhesive films were then laminated to a backing (e.g., PET and/or polyethylene), cut to the desired size, and pouched. In some instances, lauryl lactate (LL) was added to the adhesive composition at the initial mixing step. A depiction of an exemplary DMTS (dexmedetomidine transdermal system) patch of the present disclosure is provided in FIGS. 1A-C.

The DMTS and matching placebo systems were stored in original packaging until dispensed for application. Packaged DMTS/matching placebo systems were stored at controlled room temperature (15 to 30° C.).

### Example 2: Phase 2 Clinical Trial for Agitation Associated with Dementia of the Alzheimer's Type

### 1. Study Overview

The study was designed to evaluate the efficacy of DMTS on frequency and severity of agitation associated with dementia compared with placebo. This Phase 2 study will be conducted in subjects who reside in a care facility and have agitation associated with dementia of the Alzheimer's type.
Primary Objective: to evaluate the efficacy of DMTS on frequency and severity of agitation associated with dementia of the Alzheimer's type compared to placebo.
Secondary Objectives: to assess the (1) the safety and tolerability of the DMTS, including assessment of skin irritation, (2) the adhesion of the DMTS, and (3) the sedation effects of DMTS.

The primary outcome measure is change in ABS (Corrigan J D. Development of a scale for assessment of agitation following traumatic brain injury. J Clin Exp Neuropsychol. 1989; 11 (2): 261-77) score from baseline (Day −4 to Day −1 mean score) to 96 hours after application (Day 1 to Day 4).

The secondary outcome measures are:
Clinical Global Impression Scale-Severity (CGI-S) score at 96 hours post application (Day 5) relative to Pre-randomization (Day 1) baseline.
Change in ABS score from baseline (Day −4 to Day −1 mean score) to the 168 hours post first application (Day 1 to Day 7).
Change in CGI-S score at 168 hours post application (Day 8) relative to Day 1 Pre-randomization baseline.
Percentage of subjects meeting the criteria for Day 15 DTMS/placebo application.
Change from Pre-randomization Day 1 baseline score (Day −7 to Day −1 lookback) to 168 hours post application score (Day 1 to Day 7 lookback) in the Neuropsychiatric Inventory Nursing Home Version (NPI-NH).

Other secondary outcome measures are:
Incidence and severity of treatment-emergent adverse events (AEs).
Clinically important changes in safety assessment results, including, as appropriate, vital signs, change in Mini Mental Status Examination (MMSE), clinical laboratory tests, electrocardiograms (ECGs), and physical examinations (PEs).
Adhesion scores.
Sedation effect according to the Wilson sedation scale.
Severity and frequency of agitation as assessed by the Episodic Agitation Scale-Nursing Home (EAS-NH) score for DMTS compared to placebo at specified timepoints.
Clinical Global Impression Scale-Change (CGI-C) score at 96, 120, 144, and 168 hours post application for each treatment period.
Clinical Global Impression Scale-Change (CGI-C) score at 96 hours post second application (Day 19) relative to baseline (Day 1 Pre-Randomization Score) and to pre-dose (Day 15 Pre-Application score).
Change in ABS total score from baseline (Day 01 to Day −1 mean score) and from pre-dose (Day 11 to Day 14 mean score) to the 96 hours of second application (Day 15 to Day 18 mean score).
Change in CMAI total score from baseline (Day −1) to Day 15.

Phase 2 is a randomized, double-blind, placebo-controlled, two application study of DMTS or matching placebo over a 4-day treatment period (Day 1 Time 0 occurs when the first DMTS or matching placebo systems have been applied) followed 14 days later with the same treatment (or matching placebo) for an additional 4-day treatment period.

Eligible subjects will be screened up to 21 days prior to study start. Eligible subjects will be randomized 1:1:1 to treatment with 1 DMTS (1.46 mg dexmedetomidine) and 1 matching placebo, 2 DMTS (2.92 mg dexmedetomidine), or 2 matching placebos. Subjects in each treatment group will receive a total of 2 patches/systems (see Table 1) each dosing period for a total of 4 patches/systems during the study. For the second dosing period, starting on Day 15, subjects will receive the same treatment administered at the first dosing period if subject continues to meet the agitation criteria by exhibiting an ABS score of ≥22 at least once during Day 11 to Day 14. Subjects who are not eligible for Day 15 DTMS/placebo application will remain in the study without receiving any further treatment and will undergo the same assessments as the subjects receiving any further treatment and will undergo the same assessments as the subjects receiving a Day 15 DTMS/placebo application (except for PK sampling, sedation assessments, and assessments directly related to the patch, such as adhesion assessment).

Initially, 6 subjects will be enrolled and will receive DMTS/matching placebo. The safety data will be reviewed (blinded or unblinded, if necessary) and monitored from these first 6 subjects.

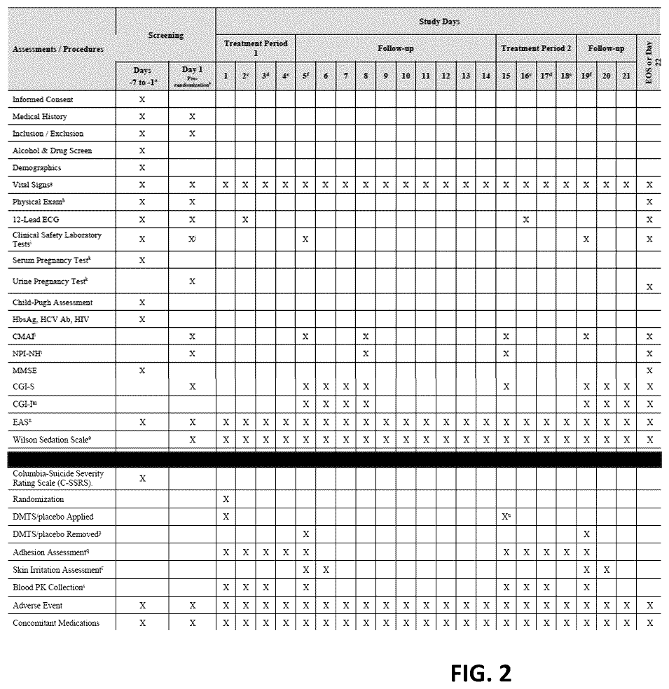

Subjects will reside in their care facility for the duration of the trial. Assessments will be performed according to the Schedule of Events (FIG. 2): agitation assessments (frequency and severity); sedation level assessments; safety assessments (vital signs including oxygen saturation [SpO₂]); DMTS/matching placebo adhesion assessments; and skin irritation assessments. In addition, blood samples will be collected for determination of plasma concentrations of dexmedetomidine

TABLE 1
Treatment Groups

Number of Systems Applied by Treatment Group

| | 1.46 mg | 2.92 mg | |
|---|---|---|---|
| System | dexmedetomidine | dexmedetomidine | Placebo |
| DTMS | 1 | 2 | 0 |
| Placebo | 1 | 0 | 2 |
| Total | 2 | 2 | 2 |

Dosing will be stopped for an individual and the DMTS removed, at any time in the trial, if the subject experiences any 1 of the following:
A Wilson sedation score of 5.
Clinically significant symptomatic bradycardia.
Clinically significant symptomatic hypotension.
A fall deemed related with the DMTS.
A severe local reaction AE at the DMTS application site.
Any SAE or severe AE.

### 2. Selection and Withdrawal of Subjects

A total of approximately 150 subjects will be randomized to treatment.

Subjects will be considered eligible to participate in this study if all the following inclusion criteria are satisfied:
Voluntarily provided written informed consent.
Male or female, residing in a care facility.
Has a diagnosis of dementia of probable Alzheimer's Disease (AD) based on National Institute on Aging and the Alzheimer's Association (NIA-AA) criteria (2018). The clinical diagnosis of “probable Alzheimer's Disease (AD)” will be based on the 2018 National Institute on Aging-Alzheimer's Association (NIA-AA) diagnostic criteria, which includes patient biomarker data as part of the research diagnosis (Jack et al., 2018). If patient biomarker data are unavailable, per the 2018 NIA-AA diagnostic criteria, the clinical diagnosis of probable AD will be based on the 2011 NIA-AA criteria (McKhann et al., 2011).
Had two or more episodes (using a 7-day lookback period) of agitation that impairs social activities, requires staff or medical intervention, or impairs ability for functional activities of daily living at Screening.
Had an ABS total score ≥22 at least once during Day −4 to Day −1 when assessing eligibility on Day 1 Pre-randomization.
Has gone a minimum of 1 week with no change in medication prior to Screening.
Has a score of ≤23 on the Mini-Mental State Examination (MMSE) at Screening.
Female subjects who are:
   Not pregnant, not lactating, and not planning to become pregnant during the study or for 1 menstrual cycle thereafter; and
   Surgically sterile; or postmenopausal (i.e. amenorrhea for >2 years as reported by subject/caregiver; postmenopausal status will be confirmed with FSH test); or had a monogamous partner who is surgically sterile; or had a same gender sex partner; or is using double-barrier contraception; or practicing abstinence; or using an insertable, injectable, transdermal, or combination oral contraceptive for 3 months prior to the study, during the study, and for 1 month following the study.
Male subjects who have female sex partners of childbearing potential must be surgically sterile or commit to use a reliable method of birth control during the study and for 1 month following the study.
Has a body weight >50 kg and a BMI of 20 to 38 kg/m², inclusive.
Able to understand the study procedures, comply with all study procedures, and agree to participate in the study program for its full duration.
Has lived in facility for at least 7 days prior to screening and will remain in facility through the completion of assessments.

Subjects will not be considered eligible to participate in this study if any one of the following exclusion criteria is met:
Has a known sensitivity to dexmedetomidine or any excipient in the DMTS/placebo.
Has a skin abnormality (e.g., scar, tattoo) or unhealthy skin condition (e.g., burns, wounds) at the DMTS/matching placebo system application site, according to examination by the investigator at screening or admission to the clinic prior to surgery.
Has a clinically significant abnormal clinical laboratory test value as determined by the investigator.
Has agitation caused by acute intoxication.
Has significant risk of suicide or homicide per investigator's assessment, or any patient with an answer of “yes” to Items 4 or 5 on the Columbia-Suicide Severity Rating Scale (C-SSRS).
Has a history of deep vein thrombosis or factor V Leiden deficiency.
Has a history of or positive test results for the human immunodeficiency virus (HIV), hepatitis B, or hepatitis C.
Has a clinically significant history or clinically significant manifestation of any of the following, as determined by the investigator: a renal, hepatic, cardiovascular, metabolic, neurologic, or psychiatric condition; congestive heart failure, peptic ulcer, gastrointestinal bleeding, or other condition that may preclude participation in the study.
Has a history of physician-diagnosed migraine, frequent non-vascular headaches (>5 per month), seizures, or taking anticonvulsants.
Has a history of syncope or other syncopal attacks.
Has present and/or significant history of postural hypotension (determined through examination by the investigator or designee), or history of severe dizziness or fainting on standing in the opinion of the investigator.
Has evidence of a clinically significant 12-lead ECG abnormality.
Has an average heart rate (HR)<60 or >100 bpm, systolic blood pressure (BP)<90 or 140 mmHg, or diastolic BP <60 or >90 mmHg, measured in 3 sequential positions (supine after 5 minutes; sitting after 2 minutes; and standing after 2 minutes) and after the sequence has been completed 3 times.
Has a history of alcohol abuse or prescription/illicit drug abuse within the previous 5 years.
Has positive results on the urine drug screen or alcohol breath test indicative of drugs of abuse or alcohol use at screening and/or clinic check-in.
Is receiving concurrent therapy that can interfere with the evaluation of efficacy or safety, such as any drug that in the investigator's opinion may exert significant synergistic interactions with dexmedetomidine.
Uses any natural health products (including chaparral, comfrey, germander, jin bu huan, kava, pennyroyal, skullcap, St. John's wort, or valerian, and excluding vitamins or mineral supplements) within 14 days prior to study drug administration and throughout the study, unless in the opinion of the investigator or designee, the product will not interfere with the study procedures or data integrity or compromise the safety of the subject. Medications with potential cardiac effects (e.g., hypotension, bradycardia) may be permissible if the subject has been stable on these medications for a minimum of 30 days prior to Screening, Medications that may cause sedation, such as quetiapine, may or may not be permissible based on the assessment of the potential for interaction with the study intervention).
Have symptoms of an upper respiratory tract infection within 14 days prior to dosing of the study drug.
Utilize oral or injectable corticosteroids within 14 days prior to dosing of the study drug (intranasal and topical corticosteroid use during this time period was allowed).
Receive any investigational product within 30 days prior to dosing of the study drug.
Has a Johns Hopkins Fall Risk Assessment Score of >13.
In the opinion of the investigator or designee, was considered unsuitable for study entry and/or unlikely to comply with the study protocol for any reason.

In addition to the inclusion and exclusion criteria listed above, respectively, each subject will agree to abide by each of the following restrictions:
Subjects cannot remove or tamper with the DMTS/matching placebo.
If the DMTS/matching placebo becomes <90% adhered, the subject will alert appropriate staff members at the clinical study unit.
Subjects are to avoid strenuous exercise, baths, saunas, steam-rooms or any other activities or environment that may lead to excessive sweating while the DMTS/matching placebo system is applied.
During treatment, subjects can take up to 1 shower daily provided the duration of the shower was limited to ≤10 minutes.
Subjects will abstain from the following foods from 1 week prior to the first dose of study medication until the last PK sample was obtained: grapefruit juice or products, pomegranate juice or products, foods containing poppy seeds, and/or drinks or foods containing quinine (e.g., tonic water) or Seville oranges (e.g., orange marmalade).
Alcohol-containing beverages will not be permitted starting 2 days prior to surgery and while housed at the clinical study unit.

Subjects will complete the study if they complete all screening procedures, receive study drug as intended in Treatment Period 1, complete the follow-up period to Day 14 and are not eligible for Treatment period 2, and complete End of Study (EOS) evaluations; or if they complete all screening procedures, receive study drug as intended in both Treatment Period 1 and 2 and complete both treatment period assessments, and complete End of Study (EOS) evaluations.

Any subject with a TEAE will be followed until the AE resolves, returns to baseline, or is deemed stable by the investigator. Any subject who voluntarily withdraws from the study (e.g., withdrawal of consent) or is discontinued for a study-related reason (e.g., as a result of a TEAE) prior to the completion of all assessments (either Treatment Period 1 or Period 2) will be considered as having discontinued early from the study.

Subjects may be discontinued from the study or decide to withdraw themselves (or by LAR) from the study under any of the circumstances listed below. The reason for early discontinuation is to be specified on the appropriate case report form (CRF).
Death
   Adverse event
   Protocol violation that warranted early discontinuation from the study as assessed by the investigator, designee, or sponsor
   Lost to follow-up
   Noncompliance
   Pregnancy
   Subject decision (withdrawal of consent when the reason was not known)
   Termination of the study by the sponsor for any reason
   Other: investigator or subject decision that cannot be classified to one of the categories above (in this case, the reason should be specified on the appropriate CRF)

### 3. Study Procedures

Subjects will be randomized in a 1:1:1 ratio of 1 DMTS (1.46 mg dexmedetomidine) and 1 matching placebo, 2 DMTS (2.92 mg dexmedetomidine), or 2 matching placebos via an interactive web response system (IWRS).

The sponsor, the investigator, personnel at the clinical study unit directly involved with monitoring and/or performing study procedures and assessments, and the subjects and informants will be blinded to treatment assignment.

The investigator can be unblinded to treatment in case of emergency if, and only if, knowledge of treatment assignment was needed to appropriately guide medical management.

Study drug (i.e., DMTS/matching placebo) will be administered by qualified and trained clinical study unit personnel. Following application, markings (such as lines) from the DMTS/matching placebo to the adjacent skin will be made using indelible ink. Visual inspection of the integrity of these markings will be performed to evaluate for any signs of tampering or removal of the DMTS or matching placebo and to assess the subject's compliance with appropriate wear.

Clinical study unit personnel will maintain an accountability log of all study drugs.

Blood samples for determination of plasma concentrations of dexmedetomidine will be obtained at the following time points for both treatment periods: pre-application and 24 hours, 48 hours, and 96 hours post-application (prior to patch removal).

The duration of study will be 36 days. Screening Period will be 14 days. Treatment Period 1 subjects will be dosed for 4 days with a follow-up for 10 days. Treatment period 2 subjects that are eligible for dosing will be dosed for 4 days with a follow-up for 3 days and 1 day for End of Study procedures, and subjects that are not eligible for dosing will be followed for 7 days and 1 day for End of Study procedures.

All pretreatment procedures outlined in this section are summarized in the Schedule of Events (FIG. 2).

### Screening (Day −21 to Day −1)

Subjects will undergo screening procedures during the Screening period. Written informed consent will be obtained before any protocol-specific procedures are performed.

The following assessments and procedures are required during screening to determine eligibility:
Signed Informed Consent Form (ICF).
ABS completed daily starting on Day −7 by site staff (will be used to assess eligibility for agitation criterion at Day 1 Pre-Randomization and to calculate ABS baseline score [Day −4 to Day −1 mean score]).
Drugs of abuse and alcohol testing.
Medical history.
Demographics.
Complete PE, including height and body weight measurements, and BMI calculation.
Review of inclusion and exclusion criteria.
Concomitant medication review.
Vital signs: BP, respiratory rate (RR), heart rate (HR), and pulse oximeter SpO₂, measured as follows: BP, RR, HR, and SpO₂ measured after the subject has been supine for at least 5 minutes, then BP and HR measured after the subject has been sitting for 1 minute, then BP and HR measured after the subject has been standing for 2 minutes. This procedure will be repeated 2 additional times to obtain BP and HR in triplicate. Oral or infrared temperature will be measured one time as well. Screening vital signs will be reviewed for any events that would exclude the subject from participating in the study (e.g., clinically significant bradycardia, arrhythmia, or other cardiovascular event).
Child-Pugh.
Serology testing (hepatitis B surface antigen [HBsAg], hepatitis C antibody [HCV Ab], and HIV). Serum pregnancy test for females of childbearing potential.
Follicle-stimulating hormone (FSH) test for females.
Clinical safety laboratory tests (hematology, serum chemistry, coagulation and urinalysis) results should be available prior to Day −7.
12-lead ECG. Screening ECG will be reviewed for any events that would exclude the subject from participating in the study (e.g., clinically significant bradycardia, arrhythmia, or other cardiovascular event).
MMSE.
EAS-NH completed daily starting on Day −7.
Columbia-Suicide Severity Rating Scale (C-SSRS).
John Hopkins Fall Risk Assessment.
Review of AEs.

### Pre-Randomization (Day 1)

Pre-randomization is the final period of Screening and will be used for Baseline. Pre-randomization procedures will be performed on Day 1 prior to randomization into a treatment group and application of study treatment. Subjects will be randomized using IWRS after the following procedures are performed and subject meets Entrance Criteria:
Review of inclusion and exclusion criteria. The subject's agitation record will be examined to determine whether the subject meets dosing criteria: two or more episodes of agitation (e.g. kicking, biting, flaying, yelling) to the point that it impairs social activities, requires staff or medical intervention, or impairs ability for functional activities of daily living. Episodes should be present during the period from Day −7 through Day −1 prior to dosing. Confirm that subject had an ABS total score ≥22 at least once during Day −1 to Day −4.
Vital signs: BP, RR, HR, and pulse oximeter SpO₂, measured as follows: BP, RR, HR, and SpO₂ measured after the subject has been supine for at least 5 minutes, then BP and HR measured after the subject has been sitting for 1 minute, then BP and HR measured after the subject has been standing for 2 minutes. This procedure will be repeated 2 additional times to obtain BP and HR in triplicate. Oral or infrared temperature will be measured one time as well. Pre-randomization vital signs will be reviewed for any events that would exclude the subject from participating in the study (e.g., clinically significant bradycardia, arrhythmia, or other cardiovascular event).
Abbreviated PE to include assessment of HEENT, heart, lungs, and skin.
Urine pregnancy test for females of childbearing potential.
12-lead ECG. Pre-randomization ECG will be reviewed for any events that would exclude the subject from participating in the study (e.g., clinically significant bradycardia, arrhythmia, or other cardiovascular event).
CGI-S.
NPI-NH.
CMAI.
Concomitant medication review.
Review of AEs.

### Treatment Procedures

### Treatment Period 1 (Day 1 to Day 14)

During the treatment period, subjects will receive a single 4-day (96-hour) application of DMTS or matching placebo. Each application will include 2 systems (patches) applied on the same side of the back. The treatment period will start (Day 1 Time 0) when the DMTS and/or matching placebo systems have been applied. For Dosing Phase 1, the patches will remain on the subject for 4 consecutive days (Day 1 through Day 4; removed on the morning of Day 5 at 96 hours post application). For Post-Dosing Phase 1, following removal of the patches after 96 hours, which is 4 days, follow-up procedures will be performed for the next 10 days (Day 5 to Day 14).

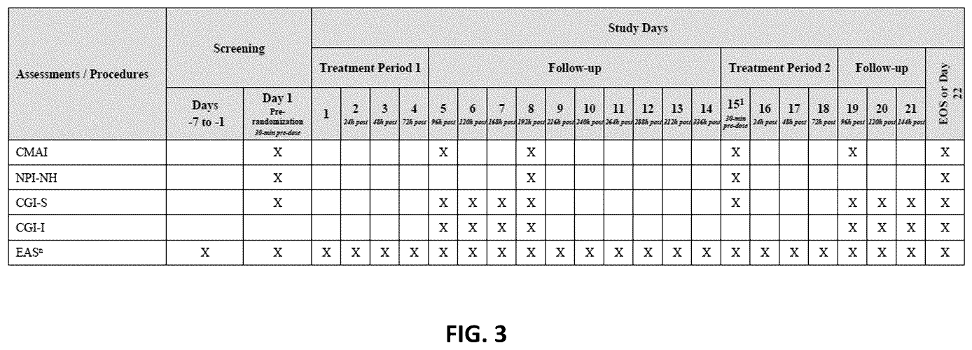

All study procedures during Treatment Period 1 are summarized in the Schedule of Events (FIG. 2), the schedule of agitation assessments (FIG. 3), the schedule of vital signs assessments (Table 2), the schedule of PK assessments (Table 3), clinical safety laboratory tests (Table 4) and schedule of sedation assessments (Table 5). When multiple procedures or assessments coincide at the same time, the sequence is: sedation, vital signs, PK, skin irritation and adhesion.

TABLE 2
Schedule of vital signs assessments.

| Day(s) | Assessment Time Points |
|---|---|
| Prior to the | Screening and Day 1 Pre-randomization^{b} |
| Treatment Period^{a} | |
| 1-14 | Pre-application and 6^{c}, 12^{c}, 16^{c}, 24, 36^{c}, 48, 60^{c}, |
| | 72, 84^{c}, 96, 104, 112^{c}, 120, 132^{c}, 144, 168, 192, |
| | 216, 240, 264, 288, and 312 hours after |
| | DMTS/matching placebo application |
| 15-21 | Pre-application/prior to Time 0 and 6^{b}, 12^{b}, 16^{b}, |
| | 24, 40, 48, 64, 72, 88, 96, 112, 120, 136, and 144 |
| | hours after DMTS/matching placebo application |
| EOS | EOS study procedures |

Resting vital signs will be obtained after the subject has been in a supine position for at least 5 minutes, then after seated for 1 minute, then after standing for 2 minutes. At screening, including pre-randomization (Day 1), the sequence of supine, seated, and standing BP and HR measurements; this sequence will be repeated for a total of 3 times. At all other time points, a single sequence of supine, seated and standing BP and HR measurements will be obtained. Only supine and sitting positions should be obtained for subjects who are wheelchair bound and unable to stand.
a. Screening and Pre-randomization (Day 1) values will be used for study eligibility.
b. Day 1 Pre-randomization values will be considered baseline.
c. At 6, 12, 16, 36, 60, 84, 112, and 132 hours after DMTS/matching placebo application/Time 0:
   If the subject has retired for the night and is asleep, no vital sign measurements will be obtained.
   If the subject is awake, supine, sitting, and standing vital sign measurements will be obtained.

The timing of vital sign assessments will be as follows:
Assessments should be performed within +30 minutes of the specified nominal time.
Assessments that coincide with a PK blood draw should be performed prior to the PK blood draw.
Assessments that coincide with patch removal should be performed prior to removal.

TABLE 3
Schedule of PK assessments.

| Day(s) | Sampling Time Points |
|---|---|
| Days 1, 2, | Pre-application and 24 hours, 48 hours, and 96 |
| 3 and 5 | hours post-application |
| Days 15, 16, | Pre-application and 24 hours, 48 hours, and 96 |
| 17 and 19 | hours post-application (only for subjects who are |
| | eligible for dosing in Treatment Period 2) |

All samples are to be obtained within +30 minutes of the specified nominal time. A total of 8 samples will be collected for PK analysis. At each collection time point, 8 mL of blood will be drawn (6 mL for PK analysis plus 2 mL for flushing the line, as needed). Based on this, a total of approximately 32 mL (subjects eligible for Treatment Period 1 dosing only) or 64 mL (subjects eligible for dosing in both treatment periods) of blood will be collected for PK. Assessments that coincide with patch removal should be performed prior to removal.

TABLE 4
Clinical safety laboratory tests.

| | hemoglobin, hematocrit, red blood cell count, white |
|---|---|
| | blood cell (WBC) count, WBC differential (absolute), |
| Hematology | and numerical platelet count |
| Serum | alanine aminotransferase, aspartate aminotransferase, |
| chemistry | albumin, alkaline phosphatase, blood urea nitrogen, |
| | calcium, carbon dioxide, chloride, cholesterol, serum |
| | creatinine, creatinine clearance (using the Cockcroft |
| | Gault formula for estimated creatinine clearance), |
| | gamma glutamyl transferase, fasting glucose, inorganic |
| | phosphorus, potassium, lactate dehydrogenase, magnesium, |
| | sodium, total bilirubin, total protein, triglycerides, and |
| | uric acid. FSH for postmenopausal females only. |
| Coagulation | Prothrombin time |
| Urinalysis | specific gravity, pH, protein, ketones, glucose, nitrite, |
| | blood, urobilinogen, and bilirubin; if nitrite, blood, or |
| | protein tests are positive, a microscopic examination |
| | will be performed |

In addition, for women of childbearing potential, pregnancy tests will be performed.

TABLE 5
Schedule of sedation assessments.

| Day(s) | Assessment Time Points |
|---|---|
| 1-14 | Pre-application, 12, 24, 36, 48, 60, 72, 96, 104, 108, |
| | 120, 132, 144, 168, 192, 216, 240, 264, 288, and 312 |
| | hours after DMTS/matching placebo application |
| 15-21 | Pre-application, 12, 24, 36, 48, 60, 72, 96, 104, 108, |
| | 120, 132, and 144 hours after DMTS/matching placebo |
| | application (only for subjects who are eligible for |
| | dosing in Treatment Period 2) |
| EOS (Day | EOS study procedures |
| 22 or EOT) | |

Assessments that coincide with patch removal should be performed prior to removal. If the subject has retired for the night and is asleep, sedation assessments will not be obtained.

### Procedures for Treatment Period 1 Dosing (Day 1 to Day 4)

The following procedures will be conducted for Treatment Period 1:

### Day 1:

Randomization using IWRS.
Vital signs at pre-application, and 6, 12, and 16 hours after application.
PK Sample prior to application,
Apply DMTS/matching placebo systems to non-hairy section of upper back within 3 hours of randomization.
EAS-NH to be completed by site staff.
ABS to be completed by site staff.
Wilson Sedation Scale at pre-application and 12 hours after application.
Adhesion Score at 6 and 12 hours after application.

### Day 2:

Vital signs at 24 and 36 hours after application.
PK Sample at 24 hours after application.
ECG.
EAS-NH to be completed by site staff.
ABS to be completed by site staff.
Wilson Sedation Scale at 24 and 36 hours after application.
Adhesion Score at 24 and 36 hours after application.

### Day 3:

Vital signs at 48 and 60 hours after application.
PK Sample at 48 hours after application.
ECG.
EAS-NH to be completed by site staff.
ABS to be completed by site staff.
Wilson Sedation Scale at 48 and 60 hours after application.
Adhesion Score at 48 and 60 hours after application.

### Day 4:

Vital signs at 72 and 84 hours after application.
EAS-NH to be completed by site staff.
ABS to be completed by site staff.
Wilson Sedation Scale at 72 hours after application.
Adhesion Score at 72 and 84 hours after application.

### Post-Dosing Period 1 (Day 5 to Day 14)

### Day 5:

Adhesion assessment at 96 hours (prior to removal).
Remove DTMS/matching placebo systems.
Swab application sites for drug residue.
Vital signs at 96 (prior to removal), 104 and 112 hours after application.
PK Sample at 96 hours after application (prior to removal).
Clinical Safety Laboratory Tests at 96 hours after application (prior to removal).
ABS to be completed by site staff.
CGI-S after patch removal.
CGI-C after patch removal.
EAS-NH to be completed by site staff.
Wilson Sedation Score at 96 (prior to removal), 104, and 108 hours after application.
Skin irritation assessment at 1 hour after DTMS/placebo system removal.

### Day 6:

Vital signs at 120 and 132 hours after application.
ABS to be completed by site staff.
CGI-S.
CGI-C.
EAS-NH to be completed by site staff.
Wilson Sedation Score at 120 and 132 hours after application.
Skin irritation assessment at 24 hours after DTMS/placebo system removal.

### Day 7:

Vital signs at 144 hours after application.
ABS to be completed by site staff.
CGI-S.
CGI-C.
EAS-NH to be completed by site staff.
Wilson Sedation Score at 144 hours after application.

### Day 8:

Vital signs at 168 hours after application.
ABS to be completed by site staff.
CGI-S.
CGI-C.
EAS-NH to be completed by site staff.
Wilson Sedation Score at 168 hours after application.

### Day 9:

Vital signs at 192 hours after application.
ABS to be completed by site staff.
EAS-NH to be completed by site staff.
Wilson Sedation Score at 192 hours after application.

### Day 10:

Vital signs at 216 hours after application.
ABS to be completed by site staff.
EAS-NH to be completed by site staff.
Wilson Sedation Score at 216 hours after application.

### Day 11:

Vital signs at 240 hours after application.
ABS to be completed by site staff.
EAS-NH to be completed by site staff.
Wilson Sedation Score at 240 hours after application.

### Day 12:

Vital signs at 264 hours after application.
ABS to be completed by site staff.
EAS-NH to be completed by site staff.
Wilson Sedation Score at 264 hours after application.

### Day 13:

Vital signs at 288 hours after application.
ABS to be completed by site staff.
EAS-NH to be completed by site staff.
Wilson Sedation Score at 288 hours after application.

### Day 14:

Vital signs at 312 hours after application.
ABS to be completed by site staff.
EAS-NH to be completed by site staff.
Wilson Sedation Score at 312 hours after application.

All study procedures during Post-Dosing Period 1 are summarized in the Schedule of Events (FIG. 2), the schedule of agitation assessments (FIG. 3) the schedule of vital signs assessments (Table 2), the schedule of PK assessments (Table 3), clinical safety laboratory tests (Table 4) and schedule of sedation assessments (Table 5).

### Treatment Period 2 (Day 15 to Day 21)

For the second treatment period, subjects will receive the same treatment administered during the first treatment period, and the patches will be placed on the opposite side of the back from the first treatment period, if the subject meets the criteria on Day 15 of an ABS total score of >22 at least once during Day 11 to Day 14.

Subjects who are not eligible for Day 15 DMTS/placebo application will remain in the study without receiving any further treatment and will undergo the same assessments as the subjects receiving Day 15 DMTS/placebo application (except for PK sampling, sedation assessments, and assessments directly related to the patch, such as adhesion assessment, etc.).

The pre-application vital signs and ECG will be reviewed for any events that would exclude the subject from continuing in the study (e.g., clinically significant bradycardia, arrhythmia, or other cardiovascular event).

Treatment Period 2 will start (Day 15, Time 0) when the DMTS or matching placebo treatment has been applied (for subjects receiving Day 15 DMTS/placebo application) or in the morning of Day 15 when ineligibility for Day 15 DMTS/placebo application has been confirmed (for subjects not receiving Day 15 DMTS/placebo application). For Dosing Phase 2, the patches will remain on the subject for 4 consecutive days (Day 15 through Day 18; removed on the morning of Day 19 at 96 hours post application). For Post-Dosing Phase 2, following removal of the patches after 4 days, follow-up procedures will be performed for the next 3 days (Day 19 to Day 21).

All study procedures during the Dosing Phase 2 and Post-Dosing Phase 2 of Treatment Period 2 are summarized in the Schedule of Events (FIG. 2). When multiple procedures or assessments coincide at the same time, the sequence is: sedation, vitals, PK, skin irritation and adhesion.

### Procedures for Treatment Period 2 Dosing (Day 15 to Day 18)

### Day 15:

The following procedures will be conducted for Treatment Period 2:
Obtain medication/kit number for the subject using IWRS (subjects receiving Day 15 DMTS/placebo application only).
Vital signs at pre-application/prior to Time 0, and 6, 12, and 16 hours after application/Time 0.
PK Sample prior to application of DTMS/matching placebo systems (subjects receiving Day 15 DTMS/placebo application only).
Apply DMTS/matching placebo systems to a non-hairy section of upper back that was not used in Treatment Period 1 (subjects received Day 15 DTMS/placebo application only).
Record time when subject was deemed ineligible for Day 15 DMTS/placebo application and define this as Time 0 for Treatment Period 2 (subjects not receiving Day 15 DMTS/placebo application only).
EAS-NH to be completed by site staff.
CMAI prior to patch application/Time 0.
ABS to be completed by site staff.
NPI-NH prior to patch application/Time 0.
CGI-S prior to patch application/Time 0.
Wilson Sedation Scale at pre-application and 12 hours after application (subjects receiving Day 15 DMTS/placebo application only).
Adhesion Score at 6 and 12 hours after application (subjects who received Day 15 DMTS/placebo application only).

### Day 16:

Vital at signs 24 and 36 hours after application/Time 0.
PK Sample at 24 hours after application (subjects who received Day 15 DMTS/placebo application only)
ECG.
EAS-NH to be completed by site staff.
ABS to be completed by site staff.
Wilson Sedation Scale at 24 and 36 hours after application (subjects who received Day 15 DMTS/placebo application only)
Adhesion Score at 24 and 36 hours after application (subjects who received Day 15 DMTS/placebo application only).

### Day 17:

Vital signs at 48 and 60 hours after application/Time 0.
PK Sample at 48 hours after application (subjects who received Day 15 DMTS/placebo application only)
EAS-NH to be completed by site staff.
ABS to be completed by site staff.
Wilson Sedation Scale at 48 and 60 hours after application (subjects who received Day 15 DMTS/placebo application only).
Adhesion Score at 48 and 60 hours after application (subjects who received Day 15 DMTS/placebo application only).

### Day 18:

Vital signs at 72 and 84 hours after application/Time 0.
EAS-NH to be completed by site staff.
ABS to be completed by site staff.
Wilson Sedation Scale at 72 hours after application (subjects who received Day 15 DMTS/placebo application only).
Adhesion Score at 72 and 84 hours after application (subjects who received Day 15 DMTS/placebo application only).

### Post-Dosing Period 2 (Day 19 to Day 21)

Unless otherwise noted, all procedures will be performed for both subjects who received Day 15 DMTS/placebo application and subjects who did not receive Day 15 treatment application.

### Day 19:

Adhesion assessment at 96 hours (prior to removal) (subjects who received Day 15 DMTS/placebo application only).
Remove DMTS/matching placebo systems (subjects who received Day 15 DMTS/placebo application only).
Swab application sites for drug residue (subjects who received Day 15 DMTS/placebo application only).
Vital signs at 96 (prior to removal), 104 and 112 hours after application/Time 0.
PK Sample at 96 hours after application (prior to removal) (subjects who received Day 15 DMTS/placebo application only).
Clinical Safety Laboratory Tests at 96 hours after application (prior to removal)/96 hours after Time 0.
EAS-NH to be completed by site staff.
ABS to be completed by site staff.
CGI-C after patch removal/96 hours after Time 0.
CGI-S after patch removal/96 hours after Time 0.
Wilson Sedation Score at 96 (prior to removal), 104 and 108 hours after application (subjects who received Day 15 DMTS/placebo application only).
Skin irritation assessment at 1 hour after DMTS/placebo system removal (subjects who received Day 15 DMTS/placebo application only).

### Day 20:

Vital signs at 120 and 132 hours after application/Time 0.
EAS-NH to be completed by site staff.
ABS to be completed by site staff.
CGI-C.
CGI-S.
Wilson Sedation Scale at 120 and 132 hours after application (subjects who received Day 15 DMTS/placebo application only).
Skin irritation assessment at 24 hours after DTMS/placebo system removal (subjects who received Day 15 DTMS/placebo application only).

### Day 21:

Vital signs at 144 hours after application/Time 0.
EAS-NH to be completed by site staff.
ABS to be completed by site staff.
CGI-C.
CGI-S.
Wilson Sedation Scale at 72 hours after application (subjects who received Day 15 DMTS/placebo application only).
Skin irritation assessment at 144 hours after DTMS/placebo system removal (subjects who received Day 15 DTMS/placebo application only).

### End of Study (Day 22 or ET)

EOS procedures will be completed on Day 22 for all subjects who complete this study. Subjects who discontinue the study early should complete EOS procedures prior to termination.

Subjects will complete the EOS procedures described below:
PE
Concomitant medication review
AE assessment
Vital signs (including oral or infrared temperature)
Clinical safety laboratory tests (hematology, serum chemistry, and urinalysis)
12-lead ECG
MMSE
Urine pregnancy test
ABS to be completed by site staff.
NPI-NH
EAS-NH to be completed by site staff.
CGI-C
CGI-S
Wilson Sedation Score

Subjects who discontinue the study drug early should be encouraged to remain in the study and complete assessments per the Schedule of Events (FIG. 2) for the respective treatment period and follow-up even after early removal of the DTMS/placebo system.

Moreover, the following assessments should be performed before removal of the DTMS:
Adhesion assessment
Vital signs
Wilson Sedation Scale
Blood sampling for PK analysis

Following early removal of the DMTS:
Skin irritation assessment at 1 and 24 hours after DMTS/placebo system removal
Continued post-dose with assessments per Schedule of Events.

DMTS/matching placebo adhesion will be assessed visually at the following time points (±15 min): 6, 12, 24, 36, 48, 60, 72, and 84 hours after each application and ≤15 minutes immediately prior to removal (96 hours after patch application). Adhesion of the DTMA/placebo system will be assessed according to the scoring system described in Table 6.

TABLE 6
DMTS Adhesion Scale

| Score | Adherence Assessment |
|---|---|
| 0 | ≥95% of the DMTS/placebo system area adhered |
| 1 | ≥90% to <95% of the DMTS/placebo system area adhered |
| 2 | ≥85% to <90% of the DMTS/placebo system area adhered |
| 3 | ≥80% to <85% of the DMTS/placebo system area adhered |
| 4 | ≥75% to <80% of the DMTS/placebo system area adhered |
| 5 | ≥70% to <75% of the DMTS/placebo system area adhered |
| 6 | <70% of the DMTS/placebo system area adhered |
| 7 | DMTS/placebo system detachment |

### Study Drug Materials and Administration

The DMTS provides extended release of dexmedetomidine over a 4-day application period. Each DMTS has a surface area of 6 cm² and contains 1.46 mg of dexmedetomidine. Excipients include lauryl lactate and an acrylate-based copolymer.

The matching placebo transdermal system is identical to the DMTS, except it does not contain dexmedetomidine.

Each DMTS or matching placebo system is individually packaged in a sealed pouch. Each pouch is labeled with study specific information, meeting all applicable regulatory requirements. All shipments of the DMTS/matching placebo systems will include a temperature monitoring device that records environmental conditions for the product at regular intervals for the entire time the shipment is in transit. Study drug will not be dispensed until the sponsor has verified that no temperature excursions occurred during shipping.

Upon receipt of the investigational products by the clinical study unit, the staff will examine the shipment to verify that the products were received in acceptable condition. Once inspected, the investigational products will be promptly transferred to the appropriate environmentally controlled storage area.

The DMTS and matching placebo systems will be stored in their original packaging until dispensed for application. Packaged DMTS/matching placebo systems will be stored at controlled room temperature.

All investigational products will be stored and handled strictly in accordance with the protocol, the package label, and the instructions supplied to the clinical study unit and its designated pharmacy.

Subjects will receive 2 DMTS/matching placebo patches (as shown in Table 1) up to 2 times. Each DMTS/matching placebo patch will be applied to a non-hairy portion of the subject's upper back by trained clinical study personnel. If the entirety of the subject's upper back is hairy, a portion of the upper back may be clipped to facilitate patch application (The application site should not be shaved). The DMTS/matching placebo patch will be worn for 4 days (96 hours from the time of application).

The following steps will be monitored to ensure proper maintenance of the DMTS/matching placebo patch:
1. The DMTS/matching placebo should not be removed or tampered with (as long as this is not necessary to address AEs or other tolerability concerns).
2. If the DMTS/matching placebo becomes <90% adhered, appropriate site staff should be alerted.
3. Subjects are to avoid strenuous exercise, baths, saunas, steam-rooms or any other activities or environment that may lead to excessive sweating while the DMTS/matching placebo systems are applied.
4. During treatment, subjects may take up to 1 shower daily, provided the duration of the shower is limited to ≤10 minutes.
5. Subjects must abstain from the following foods from 1 week prior to the first dose of study medication until the last PK sample has been obtained: grapefruit juice or products, pomegranate juice or products, foods containing poppy seeds, and/or drinks or foods containing quinine (e.g., tonic water) or Seville oranges (e.g., orange marmalade).
6. Alcohol-containing beverages will not be permitted starting 2 days prior to the first dose and during the study.

After removal, each recovered DMTS will be stored in its original pouch for drug accountability. The date of DMTS application, the subject identifier, and the application location are to be noted on the appropriate label. Specific instructions will be provided. The recovered DMTS will be returned to the sponsor.

After removal, the subject's back will be swabbed at each application the site of the DMTS application for drug residue. Using a TX714K alpha sampling snap swab (ITW Texwipe, Kernersville, NC), the swab will be dipped in a 70% isopropyl alcohol/water mixture. Excess liquid will be tapped off and the subject's back will be gently wiped where the DMTS had been applied in one direction (horizontally or vertically). The swab will be flipped over and the area of the application site will be wiped a second time using a pattern perpendicular to the first one. The break-away handle will be snapped off and the swab head will be placed into an empty glass vial (20-mL capacity or less, containing no liquid).

### 4. Assessment of Efficacy, Pharmacodynamics and Pharmacokinetics

Efficacy will be evaluated through measures of agitation frequency, severity, and improvement (FIG. 3).

### Agitated Behavior Scale

The ABS is a 14-item scale developed to allow objective assessment of agitated behavior, particularly serial assessments for the evaluation of interventions to reduce agitation. The ABS items are as follows:
1. Short attention span, easy distractibility, inability to concentrate
2. Impulsive, impatient, low tolerance for pain or frustration
3. Uncooperative, resistant to care or demanding
4. Violent and or threatening violence toward people or property
5. Explosive or unpredictable anger
6. Rocking, rubbing, moaning or other self-stimulating behavior
7. Pulling at tubes or restraints
8. Wandering from treatment areas
9. Restlessness, pacing, or excessive movement
10. Repetitive behaviors (motor or verbal)
11. Rapid, loud or excessive talking
12. Sudden changes of mood.
13. Excessive crying or laughing
14. Self-abusiveness (physical or verbal)

Behavior is rated using the following scale:
1. Absent: the behavior is not present.
2. Slight: the behavior is present but does not prevent the conduct of other, contextually appropriate behavior (the individual may redirect spontaneously, or the continuation of the agitated behavior does not disrupt appropriate behavior).
3. Moderate: the individual needs to be redirected from an agitated state to an appropriate behavior but is able to benefit from such cueing.
4. Extreme: the individual is not able to engage in appropriate behavior due to the interference of the agitated behavior, even when external cueing or redirection is provided.

For Items 10-14, a guide has been developed for scoring based on number of occurrences:

| Number of Documented Observations | |
|---|---|
| During 12 hour Shift | ABS Score |
| 0 | 1 |
| 1-2 | 2 |
| 2-6 | 3 |
| Greater than 6 | 4 |

The ABS Total Score ranges from 14 to 56. A total score of 21 or less is considered Normal; 22 to 28 is considered Mild; 29 to 35 Moderate; and 36 or more Severe Agitation. The ABS will be completed by site staff daily starting on Day −7. ABS scoring should be The ABS will be completed by site staff daily starting on Day −7. ABS scoring should be completed at approximately the same time of day each day. During each rating, the subject's behavior during the previous 24 hours since the last daily rating will be evaluated.

### Neuropsychiatric Inventory-Nursing Home Version (NPI-NH)

The Neuropsychiatric Inventory (NPI) was developed by Cummings et al. (Cummings J L, Mega M, Gray K, Rosenberg-Thompson S, Carusi D A, Gornbein J. The Neuropsychiatric Inventory: comprehensive assessment of psychopathology in dementia. Neurology. 1994 December; 44 (12): 2308-14) to assess dementia related behavioral symptoms including AD. In those instances where the caregiver is a professional (e.g., in a nursing home), the NPI-NH version of the interview is administered. The scale is administered by a certified clinician to the subject's designated informant. The informant will be a staff member of the facility who is in contact with the subject for a combined average of at least 2 hours a day for a minimum of 3 days/week and has direct observation of the subject's behavior. The NPI-NH assesses the severity and frequency of each neuropsychiatric symptom and the amount of caregiver/study partner distress engendered by each of the neuropsychiatric symptoms based on a series of scripted questions administered to the subject's designated informant with a 7-day lookback period. Based upon a positive response to the screening question, follow-up questions are read, and yes/no responses are recorded. Both frequency and severity are evaluated based upon the most abnormal behavior revealed in the follow-up questions. After frequency and severity have been determined, the informant is asked to rate the level of disruptiveness of the behavior. After frequency and severity have been determined, the caregiver is asked to rate the level of disruptiveness of the behavior.

### Frequency:

1. Rarely—less than once per week
2. Sometimes—about once per week
3. Often—several times per week but less than every day
4. Very Often—once or more per day

### Severity:

1. Mild—behavior is stressful to the resident but can be controlled
2. Moderate—behaviors are stressful for and are upsetting to the resident and are difficult to control
3. Severe—agitation is very stressful and upsetting to the resident and is very difficult or impossible to control. There is a possibility they may injure themselves and medications are often required.

### Occupational Disruptiveness:

0. Not at all
1. Minimally (almost no change in work routine)
2. Mildly (some change in work routine but little time rebudgeting required)
3. Moderately (disrupts work routine, requires time rebudgeting)
4. Severely (disruptive, upsetting to staff and other residents, major time infringement)
5. Very severely or extremely (very disruptive, major source of distress for staff and other residents, requires time usually devoted to other residents or activities).

The full NPI-NH will be administered at Pre-randomization on Day 1, on Day 8, on Day 15 prior to dosing or Time 0, and EOS (Day 22)/ET (with a 7-day lookback period for each).

### Clinical Global Impression Severity (CGI-S) and Clinical Global Impression Change (CGI-C)

The Clinical Global Impression is the rating of clinical severity and change in agitation based on the clinician's judgment. Both the CGI-S and CGI-C will be focused on the severity of agitation rather than the severity of the overall illness of dementia. The CGI-C will be referenced to the relevant CGI-S pre-application (Day 1 Pre-randomization for Treatment Period 1 and Day 15 for Treatment Period 2) and assessed at specified time point assessment. The schedule for CGI-C and CGI-S ratings is shown in FIG. 3.

The CGI-S is a 7-point (1-7) clinician-rated scale in which higher ratings indicate greater severity of agitation. The CGI-S will be rated based on severity of agitation, as follows:
0. Not assessed
1. Not at all agitated
2. Borderline agitated
3. Mildly agitated
4. Moderately agitated
5. Markedly agitated
6. Severely agitated
7. Among the most severely agitated patients

Change in agitation will be evaluated by the CGI-C. The CGI-C scores range from 1 to 7:
0. not assessed (missing),
1. much improved,
2. moderately improved,
3. mildly improved,
4. no change,
5. mildly worse,
6. moderately worse,
7. much worse.

### Episodic Agitation Scale-Nursing Home (EAS-NH)

The EAS-NH was designed to assess the frequency and severity of agitation on a daily basis. For each of the agitation behaviors, the frequency and severity are recorded for the 24-hour period. The EAS-NH provides a daily assessment of agitation frequency and severity. The EAS-NH will be completed on Day −7 through EOS. Site staff will record the frequency of agitation behaviors (i.e., Resistance, Harming Self or Others, Agitated Speech, and Restlessness/Tension) on a daily basis. For each of these agitation behaviors, the rater records the following:
A: No agitation behavior is present
B: Behavior is present, but no effect on routine, no additional attention of caregiver is needed
C: Patient less able to control behavior, some effect on routine, not needing caregiver's full attention
D: Behavior poorly controlled, has major effect on routine, needs constant caregiver attention
E: Behavior at extreme level, concern for safety.

The EAS-NH should be completed at approximately the same time of day on each day. During each rating, the subject's behavior during the previous 24 hours since the last daily rating will be evaluated. EAS-NH data can be flexibly collapsed and scaled to indicate the frequency and severity of agitation behavior during specified lookback periods (e.g., Day −4 to Day −1, Day −7 to Day −1, Day 1 to Day 4, Day 1 to Day 7, etc.).

### Cohen-Mansfield Agitation Inventory (CMAI)

The CMAI is a 29-item scale to systematically assess agitation. Institutionalized older adults are rated regarding the frequency with which they manifested physically aggressive, physically non-aggressive and verbally agitated behaviors.

The CMAI is applied using a 14-day lookback, rating the frequency of occurrence of each item over this 14-day interval on a 7-point scale as follows:
1. Never
2. Less than once a week
3. Once or twice a week
4. Several times a week
5. Once or twice a day
6. Several times a day
7. Several times an hour

The CMAI will be performed via a formal interview of a designated informant by the Investigator or designee on Day 1 (pre-randomization) and on Day 15. The informant will be a staff member of the facility or site who is in contact with the subject for a combined average of at least 2 hours a day for a minimum of 3 days/week and has direct observation of the subject's behavior.

Sedation level will be assessed by site staff at least once each day, per the Schedule of Sedation Assessments (Table 5). Sedation level will be evaluated with the Wilson Sedation Scale (Némethy M, Paroli L, Williams-Russo P G, Blanck, T J. Assessing sedation with regional anesthesia: inter-rater agreement on a modified Wilson sedation scale. Anesth. Analg. 2002; 94:723-8), presented in Table 7.

TABLE 7
Original Wilson Sedation Scale

| Score | Description |
|---|---|
| 1 | Fully awake and oriented |
| 2 | Drowsy |
| 3 | Eyes closed but rousable to command |
| 4 | Eyes closed but rousable to mild physical stimulation (earlobe tug) |
| 5 | Eyes closed and unrousable to mild physical stimulation |

Sedation assessments that coincide with patch removal should be performed prior to removal.

Cognitive Functioning will be assessed using the MMSE.

### Mini-Mental State Exam (MMSE)

The Folstein MMSE is a formal mental status examination frequently used to assess cognition. Domains measured by the MMSE include orientation to time and place, registration, attention and calculation, recall, naming, repetition, comprehension, reading, writing, and drawing. The maximum of total points on this test is 30. The MMSE will be administered during Screening and again at EOS.

### 5. Safety Assessment

Safety will include assessments of AEs (from the time informed consent is signed through the End of Study evaluations), clinical laboratory tests (hematology, serum chemistry, coagulation, and urinalysis), vital signs, ECGs, and physical examinations. Skin irritation will be evaluated using a modified Draize scoring system.

An AE is any untoward medical occurrence associated with the use of a drug in humans, whether or not considered related to the drug. An AE can be any unfavorable and unintended sign (including a clinically significant abnormal laboratory finding), symptom, or disease temporally associated with the use of a drug or investigational product, without any judgment about causality. An AE can arise from any use of the drug and from any route of administration, formulation, or dose, including an overdose.

All AEs occurring after the subject signs the ICF through the EOS visit will be recorded on the appropriate CRF. AEs will be ascertained based on volunteered signs or symptoms and by clinical observation and assessment during study visits. Spontaneously reported AEs will be recorded as such, and AEs will be elicited by clinical study unit staff using non-leading questions at designated time points. Any AE occurring at the site of DMTS/matching placebo system application will be designated on source documents and the CRF as an application site reaction.

All SAEs and unresolved AEs will be followed with appropriate medical management until resolution, return to baseline, or a stable status has been achieved as ascertained by the investigator or designee.

Pre-existing conditions, diseases, or disorders are not considered TEAEs unless there is a change in intensity, frequency, or quality following study drug administration.

Classification of adverse event (AE) severity is summarized in Table 8.

TABLE 8
Classification of Adverse Event Severity

| Classification | Definition |
|---|---|
| Mild | An event that is usually transient and requires only |
| | minimal treatment or therapeutic intervention. The |
| | event does not generally interfere with usual |
| | activities of daily living. |
| Moderate | An event that is alleviated with additional specific |
| | therapeutic intervention. The event may interfere with |
| | usual activities of daily living or cause discomfort but |
| | poses no significant or permanent risk of harm to the |
| | subject. |
| Severe | An event that requires intensive therapeutic intervention. |
| | The event interrupts usual activities of daily living or |
| | significantly affects clinical status. The event may pose |
| | a significant risk of harm to the subject and |
| | hospitalization may be required. |

For each AE, the investigator or designee will assess causality (i.e., relationship between the AE and study drug), according to the definitions provided in Table 9.

TABLE 9
Classifications for Adverse Event Causality
(Relationship to Study Drug)

| Classification | Definition |
|---|---|
| Related | There is a reasonable, evidence-based, causal relationship |
| | (eg, temporal, pharmacological, historical) between study |
| | intervention administration and the AE. |
| Not related | There is not a reasonable causal relationship between |
| | study intervention administration and the AE. The |
| | investigator should state the most likely alternative |
| | causality explanation (eg, underlying illness, concomitant |
| | medication). |

An SAE is defined as an AE that resulted in any of the following outcomes:
Death
A life-threatening AE (i.e., the AE placed the subject at immediate risk of death; an AE that hypothetically might have caused death if it were more severe was not an SAE by this criterion)
An in-patient hospitalization or prolongation of an existing hospitalization
A persistent or significant incapacity or substantial disruption of the ability to conduct normal life functions
A congenital anomaly/birth defect
An important medical event that may not have resulted in death, may not have been life-threatening, or may not have required hospitalization, but based on appropriate medical judgment, may have jeopardized the subject or may have required medical or surgical intervention to prevent the outcomes listed above.

Skin irritation at each DMTS/matching placebo site will be assessed 1 hour (±5 min) and 24 hours (±2 h) after removal of the DMTS/matching placebo. Skin irritation assessment(s) will include an evaluation of the presence and severity of erythema, edema, papules, and vesicles (if any) using the scale in Table 7.

Whenever a skin irritation assessment results in a non-zero score, the symptom will be documented and tracked as a TEAE. Any subject with a skin irritation score of >1 will be followed through resolution or stabilization.

Any skin irritation or other local effect at the study drug application site that is not included in the skin irritation scale (Table 10) and/or is identified outside of the scheduled skin irritation assessment time points will be recorded as a TEAE and designated as an application site reaction.

TABLE 10
Skin Irritation Scoring Scale

| Score | Erythema | Edema | Papules | Vesicles |
|---|---|---|---|---|
| 0 | None | None | None | None |
| 1 | Minimal/Mild | Minimal/Mild | Minimal/Mild | Minimal/Mild |
| | (diffuse, barely | (barely visible; | (<10 seen per | (<10 seen per |
| | visible, locally | skin elevated <1 | application area; | application area; |
| | restricted) | mm) | non-confluent) | nonconfluent) |
| 2 | Moderate | Moderate (skin | Moderate (≥10 | Moderate (≥10 |
| | (definite | elevated~1 mm) | observed on <50% | observed on <50% |
| | redness) | | of application | of application |
| | | | area; ±increasing | area; ±increasing |
| | | | confluence) | confluence) |
| 3 | Severe | Severe (skin | Severe (≥10 | Severe (≥10 |
| | (strong fiery | raised >1 mm) | observed on ≥50% | observed on ≥50% |
| | redness; may | | of application | of application |
| | spread beyond | | area; ±increasing | area; ±increasing |
| | local application | | confluence) | confluence) |
| | area) | | | |

Clinical safety laboratory tests will include hematology, fasting, serum chemistry, coagulation, and urinalysis, as described in Table 4. Hematology, serum chemistry, coagulation, and urinalysis will be performed at screening While the Screening period will extend from Day −14 through Day 1 Pre-randomization, screening clinical safety laboratory tests must be performed within 7 days prior to Pre-randomization—Day 1 (i.e., no earlier than Day −7), as well as early enough that results will be available on Day 1 in time for randomization. Hematology, serum chemistry and urinalysis will also be performed at Day 5, Day 19, and at the EOS visit or at the time of early discontinuation from the study. FSH test will be performed at screening for postmenopausal females only.

Results of all laboratory tests will be reviewed by the investigator or designee. For clinical safety laboratory testing, up to approximately 71 mL of blood will be obtained from each subject. Additional samples may be taken at the discretion of the investigator or designee if the results of any tests fall outside reference ranges or if clinical symptoms necessitate additional testing to ensure safety.

The total volume of blood that will be drawn during this study for hematology, serum chemistry, coagulation serology, and PK sampling is approximately 135 mL per subject.

At screening only, blood will be collected according to standard procedures for a viral serology test to screen for HBsAg, HCV Ab, and HIV. Counseling will be provided to the subject both before and after testing for hepatitis and HIV. Only subjects with negative viral serology tests will be eligible for the study.

Tests for alcohol and drugs of abuse will be performed on site at screening. Only subjects with negative results to both tests will be eligible for the study. The results of the tests will be recorded in source documentation. Urine will be tested for opiates, amphetamines, cocaine, benzodiazepines, and cannabinoids. Breath testing will be performed for alcohol.

All medications (prescription and non-prescription, herbal medications, or investigational drugs) taken by the subject during the 30 days prior to screening and prior to dosing will be recorded in the source documentation. The reported medications will be reviewed and evaluated by the investigator or designee to determine if they affect the subject's eligibility to participate in the study.

For female subjects of childbearing potential, a serum pregnancy test will be performed during screening, and a urine pregnancy test will be performed at Pre-randomization on Day 1 and EOS. Subjects with positive pregnancy tests will be excluded from the study.

All medications (prescription and non-prescription, herbal medications, or investigational drugs) taken by the subject during the 30 days prior to screening and prior to dosing will be recorded in the source documentation. The reported medications will be reviewed and evaluated by the investigator or designee to determine if they affect the subject's eligibility for continued participation in the study.

All medications taken by the enrolled subjects from screening through the EOS will be documented and recorded on the appropriate CRF. The reported medications will be reviewed and evaluated by the investigator or designee to determine if they affect the subject's eligibility for continued participation in the study.

Medications with potential cardiac effects (e.g., hypotension, bradycardia) may be permissible if the subject has been stable on them for a minimum of 30 days prior to Screening.

Medications that may cause sedation, such as quetiapine (Seroquel), may or may not be permissible based on the assessment of the Investigator and the Medical Monitor of the potential for interaction with the study intervention.

Subjects may receive rescue medication for agitation. Standard of care for treatment of agitation may be used per investigator judgment. All rescue medications taken by the enrolled subjects from Screening through EOS will be documented and recorded on the appropriate CRF.

The following therapies will be prohibited within the specified screening period and during the study:
Chronic opioid therapy for >2 weeks within the month prior to study drug administration.
Natural health products (including chaparral, comfrey, germander, jin bu huan, kava, pennyroyal, skullcap, St. John's wort, or valerian, and excluding vitamins or mineral supplements) within 14 days prior to study drug administration and throughout the study, unless in the opinion of the investigator or designee, the product will not interfere with the study procedures or data integrity or compromise the safety of the subject.
Opiates, amphetamines, cocaine, benzodiazepines, and cannabinoids as determined by positive urine drug screen test result.
Concurrent therapy that could interfere with the evaluation of efficacy or safety, such as any drugs that in the investigator's opinion may exert significant calming properties or act synergistically with the study drug. As-needed treatment with medications that may cause sedation may or may not be permissible based on the assessment of the Investigator of the potential for interaction with the study intervention.

A PE (excluding breast, genitourinary, and rectal exam) will be performed to assess the subject's overall health and physical condition at screening and at the EOS visit. The PE will include assessment of the following body systems: head, ears, eyes, nose, and throat (HEENT), lymph nodes, heart, lungs, extremities, peripheral vascular, abdomen, neurological, chest/back, and skin. At the screening visit only, the PE will also include height and weight measurements (and calculation of BMI).

An abbreviated PE will be performed at clinic check-in and will include assessments of the following body systems: HEENT, heart, lungs, and skin.

Any new or worsened physical finding (observed following administration of the DMTS/matching placebo) that meets the definition of an AE should be reported as an AE.

A 12-lead ECG will be obtained at screening, clinic check-in, and EOS. Each ECG will be obtained after the subject has been resting for at least 5 minutes in a supine position. The ECG will electronically measure and calculate ventricular rate and the PR, QRS, QT, and QTc intervals. The investigator will review all ECG findings to assess whether any values outside the respective normal ranges are clinically significant. All clinically significant abnormal findings that meet the definition of an AE are to be reported as AEs and recorded on the AE CRF.

Vital signs, consisting of BP, respiratory rate (RR), heart rate (HR), and SpO₂, will be measured according to the schedules provided in FIG. 2 and Table 2. Resting vital signs will be obtained after the subject has been in a supine position for at least 5 minutes, then after seated for 1 minute, then after standing for 2 minutes. At screening, including pre-randomization (Day 1), the sequence of supine, seated, and standing BP and HR measurements; this sequence will be repeated for a total of 3 times. At all other time points, a single sequence of supine, seated and standing BP and HR measurements will be obtained.

Oral or infrared body temperature will be measured at screening, pre-randomization (Day 1), and EOS.

Subjects should be advised to be cautious when rising from a supine or seated position in case of orthostatic hypotension symptoms (dizziness, postural instability, feeling faint, etc.). In general, it is recommended that subjects transition slowly from the supine position to a seated position or from the seated position to the standing position. While seated, subjects should have their legs dangling and not crossed. While arising to the standing position, subjects should avoid holding their breath. If a subject becomes lightheaded or otherwise symptomatic of orthostatic hypotension upon arising or during the initiation of the standing BP measurement, the subject is to immediately return to the supine position and remain supine until the symptoms subside. A repeat supine BP measurement should be obtained.

If the timing of vital sign measurements coincides with PK sampling times, the vital signs measurements should be obtained prior to the PK blood draw.

Vital signs will also be measured and recorded in the event of a symptomatic AE potentially associated with changes in vital signs. Any clinically significant abnormal vital sign(s) will be recorded on the AE CRF.

Clinically significant bradycardia in a given subject is defined as either an absolute HR <45 bpm or a drop of >30% relative to the mean HR value obtained prior to DMTS/matching placebo application, and this value is sustained for 1 minute.

Clinically significant symptomatic bradycardia will be accompanied by one or more of the following sustained symptoms: syncope, dizziness, fatigue, chest pain, difficulty breathing, hypoxemia (SpO₂<90%).

Clinically significant hypotension in a given subject is defined as the lower of either an absolute BP value (systolic BP <80 mmHg or diastolic BP <50 mmHg) or a drop of >30% relative to the respective mean BP value obtained prior to DMTS/matching placebo application, and this value (absolute value or drop relative to baseline) is sustained for 1 minute.

Clinically significant symptomatic hypotension will be accompanied by one or more of the following sustained symptoms: syncope, dizziness, fatigue, chest pain, difficulty breathing, hypoxemia (SpO₂<90%), cold clammy skin.

If the subject experiences a symptomatic clinically significant cardiovascular event (such as bradycardia or hypotension), the DMTS should be removed, and the following may be implemented as necessary per investigator judgment:
Administer fluids (oral or IV).
Administer small doses of atropine if the subject is both hypotensive and bradycardic.
Administer a vasopressor if the subject is hypotensive.

### Columbia-Suicide Severity Rating Scale (C-SSRS)

The Columbia Suicide Severity Rating Scale (C-SSRS) is a suicidal ideation rating scale. The scale identifies behaviors and thoughts that are associated with an increased risk of suicidal actions in the future. The C-SSRS assessment will be conducted at Screening.

### Johns Hopkins Fall Risk Assessment

The Johns Hopkins Fall Risk Assessment Tool helps staff determine if a subject is at high or low risk of falls. The scale uses various point-based criteria to derive a total risk score. Scores of <6 are considered reflective of a low fall risk, 6-13 a moderate fall risk, and >13 a high fall risk. The Johns Hopkins Fall Risk Assessment will be conducted at Screening. Subjects with Johns Hopkins Fall Risk Assessment scores of >13 will be excluded from the study.

### Child-Pugh Hepatic Impairment Assessment

The Child-Pugh score is a measure calculated based on multiple clinical measures of liver disease to determine an overall assessment of hepatic impairment. The Child-Pugh score will be determined during Screening. Subjects with Child-Pugh scores of >5 will be excluded from the study.

### 6. Assessment of Efficacy, Pharmacokinetic and Pharmacodynamic Data

The primary efficacy outcome measure is change in ABS total score from baseline (Day −4 to Day −1 mean score) to the 96 hours of first treatment application (Day 1 to Day 4). The estimate of the primary efficacy outcome measure is the difference in the mean changes from baseline (Day −4 to Day −1 mean score) for the 96 hours of first treatment application (Day 1 to Day 4) in ABS total score between subjects randomized to placebo and those randomized to a DMTS dose group in the ITT population with the treatment policy strategy applied to intercurrent events of interest such as non-compliance, discontinuation of treatment or use of prohibited medication, etc. The analysis of the primary efficacy outcome measure will be conducted using a mixed model for repeated measures (MMRM) based on a restricted maximum likelihood method with change from baseline at each day of first treatment application as the response variable, including treatment group, baseline, study center, day, baseline-by-day interaction, and treatment group-by-day interaction as model terms. The population level summary will be the difference in equally weighted treatment differences across Days 1 through 4 from the mixed model.

Similarly, the ITT population will be the target population for assessments of the key secondary outcome measures and the treatment policy strategy will be applied to the occurrence of intercurrent events of interest. The change from baseline (Day −4 to Day −1 mean score) in ABS scores through 168 hours post first treatment application (Day 1 to Day 7) will be analyzed using the same MMRM as for the primary outcome measure. The CGI-S score at 96 hours post application relative to baseline (Day 1 Pre-randomization score) and the CGI-S score at 168 hours post application relative to baseline (Day 1 Pre-randomization score) will be analyzed similarly. The change in NPI-NH score from Day 1 Pre-randomization baseline to 168 hours post application will be analyzed using an ANCOVA model with treatment, Day 1 pre-randomization score, and study center as model terms. The methodology for the analysis of the percentage of subjects meeting the criteria for Day 15 DTMS/placebo application, any supplemental analyses of agitation using the ABS, CGI-S, CGI-C, NPI-NH, and CMAI at additional time points during Treatment Period 1 and during Treatment Period 2, as well as methods to control Type I error associated with multiplicity of testing will be specified in the SAP.

Venous blood samples for PK analyses will be obtained according to the schedule described in Table 3. A total of 8 PK sampling time points are planned as follows: pre-application and 24 hours, 48 hours, and 96 hours post-application for Treatment Periods 1 and 2. The 96-hour sample collections will be performed prior to parch removal. All samples are to be obtained within +30 minutes of the specified nominal time. Treatment Period 2 PK samples will not be collected from subjects that are not eligible for dosing in Treatment Period 2.

Approximately 8 mL of blood will be drawn (6 mL for PK analysis plus 2 mL for flushing the line, as needed) for each PK sample. As such, a total of up to approximately 32 mL (subjects eligible for Treatment Period 1 dosing only) or 64 mL (subjects eligible for dosing in both treatment periods) of blood per subject will be drawn for PK assessments.

Plasma concentrations of dexmedetomidine at each time point will be summarized using the arithmetic and geometric mean, SD, median, range, and coefficient of variation. The time-course of plasma concentration data will be plotted as appropriate.

Assessment of sedation scores over time will be summarized.

### 7. Analysis of Safety Data

Clinical safety laboratory tests will include hematology, fasting serum chemistry, coagulation, and urinalysis. Hematology, serum chemistry, coagulation, and urinalysis will be performed at screening. While the Screening period will extend from Day −21 through Day 1 Pre-randomization, screening period laboratory tests should be performed and results available prior to the start of the ABS screening assessments that take place starting on Day −7 through Day −1. Hematology, serum chemistry and urinalysis will also be performed at Day 5, Day 19, and at the EOS visit or at the time of early discontinuation from the study. FSH test will be performed at screening for post-menopausal females only.

Results of all laboratory tests will be reviewed by the investigator or designee. For clinical safety laboratory testing, up to approximately 71 mL of blood will be obtained from each subject. Additional samples may be taken at the discretion of the investigator or designee if the results of any tests fall outside reference ranges or if clinical symptoms necessitate additional testing to ensure safety. The total volume of blood that will be drawn during this study for hematology, serum chemistry, coagulation, serology, and PK sampling is approximately 135 mL per subject.

All subjects who receive study treatment will be included in the summaries and listings of safety data. AEs will be coded using the Medical Dictionary for Regulatory Activities (MedDRA) and tabulated by system organ class (SOC) and preferred term (PT). Individual events (PT) within each SOC will be presented in order of descending frequency. AEs will be presented by maximum severity and by relationship to the study treatment. SAEs will be summarized separately.

Descriptive statistics (mean, median, SD, and range) as well as change from baseline will be presented for vital signs, MMSE, ECG data, and clinical laboratory data over time. For clinical laboratory data, the number (percentage) of subjects with results below, within, and above normal ranges will be tabulated by visit. The quantitative change from baseline will be summarized and shift tables indicating change from normal status will be presented, if appropriate. Abnormal findings in laboratory data will be listed.

Termination data, including reason for early discontinuations, will be summarized overall and by treatment. Concomitant medications, PE findings, and medical history will be listed. Skin irritation data will be summarized. Adhesion scores at each assessment time point will be summarized.

During screening, the Estimated Glomerular Filtration Rate (eGFR) should be calculated. For study eligibility, the subject's eGFR should be above the lower limit of the age- and gender-specific range, shown in Table 11. The ranges in this table were obtained from approximately 3000 potential kidney donors ranging from 20 to 80 years of age.

TABLE 11
Age- and Gender-Specific Normal GFR Reference Ranges

| | Measured GFR (mL/min/1.73 m²) | | | | | |
|---|---|---|---|---|---|---|
| | Male | | | Female | | |
| | Lower | | Upper | Lower | | Upper |
| Age (years) | Limit | Mean | Limit | Limit | Mean | Limit |
| 20-34 | 74 | 100 | 127 | 72 | 99 | 125 |
| 35 | 73 | 100 | 126 | 72 | 99 | 125 |
| 40 | 70 | 96 | 123 | 68 | 95 | 121 |
| 45 | 67 | 93 | 119 | 64 | 91 | 117 |
| 50 | 63 | 90 | 116 | 60 | 87 | 114 |
| 55 | 60 | 86 | 113 | 56 | 83 | 110 |
| 60 | 57 | 83 | 110 | 52 | 79 | 106 |
| 65 | 53 | 80 | 106 | 48 | 75 | 102 |
| 70 | 50 | 77 | 103 | 44 | 71 | 98 |
| 75 | 47 | 73 | 100 | 40 | 67 | 94 |
| 80 | 43 | 70 | 97 | 36 | 63 | 90 |

GFR = glomerular filtration rate
Note:
The ranges in this table are based on measured GFRs from 2974 prospective living kidney donors.

### 8. Statistics

Complete details of the statistical analyses will be documented in the Statistical Analysis Plan (SAP), which will include details of estimates for key efficacy outcome measures, analysis populations, summary strategies, sensitivity analyses, and any changes to the proposed analyses listed here, if applicable.

### Determination of Sample Size

Approximately 150 subjects are planned to be randomized in the study. With 1:1:1 randomization to treatment groups (1 DMTS 1.46 mg dexmedetomidine and 1 matching placebo, 2 DMTS 2.92 mg dexmedetomidine, or 2 matching placebos), a sample size of approximately 47 evaluable subjects per treatment group is required to achieve at least 85% power to detect a 2.0-point difference in change from baseline of ABS total score from baseline to the 96 hours of first application (Day 1 to Day 4) between either DMTS group and the placebo group. This calculation is based on an assumed two-sample t-test with two-sided alpha set at the 0.05 level, and a common standard deviation (SD) of change scores of 3.2 points. Assuming a 6% dropout rate, approximately 150 subjects (50 subjects per treatment group) will be required to be randomized to retain 141 evaluable subjects.

### Randomization

An IWRS will be used to randomize subjects equally to a treatment group.

### Analysis Populations

The Intent to Treat (ITT) population consists of all subjects who are randomized. The ITT population will be the target population for primary and key secondary efficacy assessments, where the treatment group as randomized will be used for analyses. A subset of the ITT population will include all subjects receiving a second application of treatment. This subset will be the target population for select exploratory assessments focusing on efficacy associated with the second application of treatment.

The Safety population includes all randomized and treated subjects. The Safety population will be used for summarizing safety data according to the treatment received.

Statistical analyses will be performed using SAS.

Demographic and other baseline characteristics at study entry will be summarized for all subjects. Continuous variables will be summarized with mean, SD, median and range; categorical characteristics will be presented by percentage of subjects belonging to each relevant category.

The study will demonstrate reduction in agitation in patients via one or more metrics following treatment. The study will also demonstrate that patients are not substantially sedated by treatment.

### 6. EQUIVALENTS AND INCORPORATION BY REFERENCE

While the provided disclosure has been particularly shown and described with reference to a preferred embodiment and various alternate embodiments, it will be understood by persons skilled in the relevant art that various changes in form and details can be made therein without departing from the spirit and scope of the provided disclosure.

All references, issued patents, and patent applications cited within the body of the instant specification, including U.S. Provisional Application No. 63/698,171, are hereby incorporated by reference in their entirety, for all purposes.

1. A method of treating agitation in a subject in need thereof, comprising applying one or more transdermal delivery patches to a skin surface of the subject, who is not agitated at the time of application, wherein:
each transdermal delivery patch comprises a pharmaceutical composition comprising dexmedetomidine;
the one or more transdermal delivery patches deliver a therapeutically effective non-sedating amount of dexmedetomidine to the subject;
each transdermal delivery patch comprises a drug layer affixed to a backing layer, wherein the drug layer comprises from 1 mg to 3.5 mg dexmedetomidine, a pressure sensitive adhesive comprising a hydroxyl functionalized acrylate polymer, and lauryl lactate; wherein:
the drug layer has an adhesive surface suitable for adhesion to a skin surface, wherein the adhesive surface has a surface area from 3 cm² to 20 cm²; and
the backing layer comprises polyethylene and polyethylene terephthalate;
the subject has an Agitated Behavior Scale (ABS) score of at least 22 at least once when assessed on one of the four days prior to application of the one or more transdermal delivery patches; and
the subject's ABS score 96 hours following application and maintenance of the one or more transdermal delivery patches on the skin surface of the subject is reduced compared to the subject's baseline ABS score assessed prior to application of the one or more transdermal delivery patches.

2-81. (canceled)

82. The method of claim 1, wherein the agitation is associated with dementia of the Alzheimer's type.

83. The method of claim 1, wherein the subject has a diagnosis of dementia of probable Alzheimer's Disease.

84. The method of claim 1, wherein the subject has a Mini-Mental State Exam (MMSE) score of ≤23 when administered on one of the 7 days prior to applying the one or more transdermal patches.

85. The method of claim 1, wherein the subject's ABS score 168 hours following application and maintenance of the one or more transdermal delivery patches on the skin surface of the subject for 4 days is reduced compared to the subject's baseline ABS score assessed prior to application of the one or more transdermal delivery patches.

86. The method of claim 1, wherein the subject's Cohen Mansfeld Agitation Inventory (CMAI) total score 15 days after application of the one or more transdermal delivery patches to the skin surface of the subject for 4 days is reduced compared to the subject's baseline CMAI total score.

87. The method of claim 1, wherein the subject's Neuropsychiatric Inventory-Nursing Home Version (NPI-NH) score 168 hours following application and maintenance of the one or more transdermal delivery patches on the skin surface of the subject for 4 days is reduced compared to the subject's baseline NPI-NH score.

88. The method of claim 1, wherein subject's Episodic Agitation Scale-Nursing Home (EAS-NH) score 24 hours following application and maintenance of the one or more transdermal delivery patches on the skin surface of the subject for 4 days is reduced compared to the subject's baseline EAS-NH score assessed prior to application of the one or more transdermal delivery patches.

89. The method of claim 1, wherein the subject's Clinical Global Impression Scale-Change (CGI-C) score 96 hours following application and maintenance of the one or more transdermal delivery patches on the skin surface of the subject for 4 days is reduced compared to the subject's baseline CGI-C score measured prior to application of the one or more transdermal delivery patches.

90. The method of claim 1, wherein the subject's Clinical Global Impression Scale-Severity (CGI-S) score 96 hours following application and maintenance of the one or more transdermal delivery patches on the skin surface of the subject for 4 days is reduced compared to the subject's baseline CGI-S-score measured prior to application of the one or more transdermal delivery patches.

91. The method of claim 1, wherein the one or more transdermal delivery patches each comprise from 1 mg to 2 mg dexmedetomidine.

92. The method of claim 1, wherein the one or more transdermal delivery patches each comprise 1.46 mg dexmedetomidine.

93. The method of claim 1, wherein the one or more transdermal delivery patches each comprise from 2.5 mg to 3.5 mg dexmedetomidine.

94. The method of claim 1, wherein the one or more transdermal delivery patches each comprise 2.92 mg dexmedetomidine.

95. The method of claim 1, wherein the one or more transdermal delivery patches collectively comprise from 2.5 mg to 3.5 mg dexmedetomidine.

96. The method of claim 1, wherein the one or more transdermal delivery patches collectively comprise 2.92 mg dexmedetomidine.

97. The method of claim 1, wherein the adhesive surface has a surface area from 6 cm² to 7 cm².

98. The method of claim 97, wherein the adhesive surface has a surface area of 6 cm².

99. The method of claim 1, wherein the adhesive surface has a surface area from 12 cm² to 13 cm².

100. The method of claim 99, wherein the adhesive surface has a surface area of 12 cm².

101. The method of claim 1, wherein the transdermal delivery device further comprises a release liner adhered to the adhesive surface of the drug layer.

102. The method of claim 1, wherein the one or more transdermal delivery patches provide an in vitro average flux, when measured on human cadaver skin, from 0.005 μg/cm²/hr to 5 μg/cm²/hr 24 hours after application of the one or more transdermal delivery patches to a skin surface and maintenance of contact between the skin surface and the one or more transdermal delivery patches.

103. The method of claim 1, wherein the one or more transdermal delivery patches provide an in vitro dexmedetomidine peak flux, when measured on human cadaver skin, from 0.1 μg/cm²/hr to 3 μg/cm²/hr.

104. The method of claim 1, wherein:
the agitation is associated with dementia of the Alzheimer's type;
the one or more transdermal delivery patches each comprise from 1 mg to 2 mg dexmedetomidine; and
the adhesive surface has a surface area from 6 cm² to 7 cm².

105. The method of claim 104, wherein the one or more transdermal delivery patches each comprise 1.46 mg dexmedetomidine.

106. The method of claim 105, wherein the surface area is 6 cm².

107. The method of claim 1, wherein:
the agitation is associated with dementia of the Alzheimer's type;
the one or more transdermal delivery patches each comprise from 2.5 mg to 3.5 mg dexmedetomidine; and
the adhesive surface has a surface area from 12 cm² to 13 cm².

108. The method of claim 107, wherein the one or more transdermal delivery patches each comprise 2.92 mg dexmedetomidine.

109. The method of claim 108, wherein the surface area is 12 cm².

110. A method of treating agitation in a subject in need thereof, comprising applying one or more transdermal delivery patches to a skin surface of the subject, who is not agitated at the time of application, wherein:
each transdermal delivery patch comprises a pharmaceutical composition comprising dexmedetomidine;
the one or more transdermal delivery patches deliver a therapeutically effective non-sedating amount of dexmedetomidine to the subject;
each transdermal delivery patch comprises a drug layer affixed to a backing layer, wherein the drug layer comprises from 1 mg to 3.5 mg dexmedetomidine, a pressure sensitive adhesive comprising a hydroxyl functionalized acrylate polymer, and lauryl lactate; wherein:
the drug layer has an adhesive surface suitable for adhesion to a skin surface, wherein the adhesive surface has a surface area from 3 cm² to 20 cm²; and
the backing layer comprises polyethylene and polyethylene terephthalate;
the subject has a diagnosis of dementia of probable Alzheimer's Disease; and
the subject's ABS score 96 hours following application and maintenance of the one or more transdermal delivery patches on the skin surface of the subject is reduced compared to the subject's baseline ABS score assessed prior to application of the one or more transdermal delivery patches.
